(19) United States
(12) Patent Application Publication
(10) Pub. No.: US 2026/0191858 A1
(43) Pub. Date: Jul. 9, 2026

(54) COMBINATION THERAPIES

(71) Applicant: Kallyope, Inc., New York, NY (US)

(72) Inventors: Shirly PINTO, Ridgewood, NJ (US); Iyassu SEBHAT, Jersey City, NJ (US); Brett LAURING, Tenafly, NJ (US); Nancy A. THORNBERRY, Southport, CT (US)

(21) Appl. No.: 18/874,815

(22) PCT Filed: Jun. 20, 2023
(86) PCT No.: PCT/US2023/068729

§ 371 Date: Dec. 13, 2024

Related U.S. Application Data
(60) Provisional application No. 63/355,258, filed on Jun. 24, 2022.

(51) Int. Cl. A61K 31/506; A61K 31/155; A61K 31/522; A61K 31/662; A61K 31/675; A61K 31/7064; A61P 3/04

(52) U.S. Cl. CPC A61K 31/506; A61K 31/155; A61K 31/522; A61K 31/662; A61K 31/675; A61K 31/7064; A61P 3/04

(57) ABSTRACT

The present invention relates to pharmaceutical combinations comprising GRP119 agonist compounds of the presently-defined Formula (A) and GRP40 agonist compounds of the presently-defined Formula (B), and their use for the treatment of conditions or disorders involving the gut-brain axis. In some embodiments, the condition or disorder is a metabolic disorder, such as diabetes, obesity, or nonalcoholic steatohepatitis (NASH); a nutritional disorder, such as short bowel syndrome; or an eating disorder, such as binge eating disorder.

### CROSS-REFERENCE

This application claims the benefit of U.S. Provisional Application Ser. No. 63/355,258 filed Jun. 24, 2022, which is hereby incorporated by reference in its entirety.

### BRIEF SUMMARY OF THE INVENTION

Disclosed herein, in certain embodiments, combination therapies useful for the treatment of conditions or disorders involving the gut-brain axis comprising a GPR119 agonist and a GPR40 agonist compound. In some embodiments, the GPR119 and/or GPR40 agonists are gut-restricted or selectively modulate GPR119 and/or GPR40 located in the gut. In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, binge eating disorder, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

Disclosed herein is a method of treating a condition or disorder involving the gut-brain axis in an individual in need thereof, the method comprising administering to the individual:
i) a compound of Formula (A):

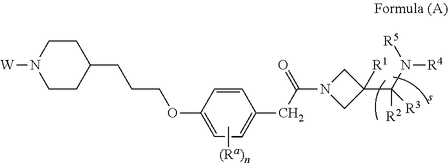

or a pharmaceutically acceptable salt, solvate, or stereoisomer thereof, wherein:
R¹ is hydrogen, —OH, or C₁₋₈ alkyl, wherein the alkyl is unsubstituted or substituted by —OH or —O(C₁₋₆ alkyl);
each R² and R³ is hydrogen;
or R² and R³ on the same carbon atom are taken together to form ═O;
R⁴ is hydrogen or C₁₋₆ alkyl;
R⁵ is C₁₋₈ alkyl, C₃-8 cycloalkyl, 4- to 8-membered heterocycloalkyl, —[(CH₂)ᵣ—Z]ₜ—R⁶, —[(CHR^{d})ᵣ—Z]ₜ—R⁶, or —[(C(R^{d})₂)ᵣ—Z]ₜ—R⁶; wherein each alkyl, cycloalkyl, and 4- to 8-membered heterocycloalkyl is substituted by 1-6 R^{c} groups
   each Z is independently —CH₂O—, —CH₂NR^{d}, —CH₂N⁺(R^{d})₂—, or —NH—C(═O)—NH—;
   each r is independently 1-6;
   each t is independently 1-6;
   R⁶ is hydrogen, C₁₋₈ alkyl, C₃₋₈ cycloalkyl, or 4- to 8-membered heterocycloalkyl, wherein the alkyl, cycloalkyl, or 4- to 8-membered heterocycloalkyl is unsubstituted or substituted by 1-6 R^{c} groups;
or R⁴ and R⁵ are taken together with the nitrogen to which they are attached to form a 4- to 8-membered heterocycloalkyl, which is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH) 2, —P(═O)(OH)(R^{d}), —P(═O)(OH)(OR^{d}),

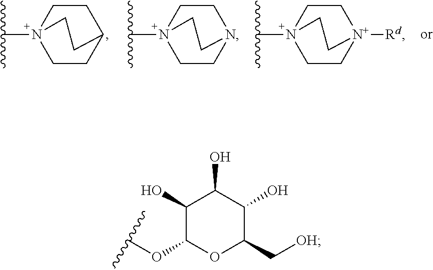

each R^{d} is independently C₁₋₆ alkyl;
each R^{a} is independently halogen, —CN, C₁₋₆ alkyl, C₁₋₆ fluoroalkyl, or C₃₋₆ cycloalkyl;
W is phenyl or 5-6 membered monocyclic heteroaryl, wherein the phenyl or heteroaryl is unsubstituted or substituted with 1, 2, or 3 substituents selected from R^{b};
   each R^{b} is independently halogen, —OH, —CN, —C(O)OH, —C(O)O(C₁₋₆ alkyl), C₁₋₆ alkyl, C₁₋₆ alkoxy, C₃-6 cycloalkyl, phenyl, or 5- to 6-membered heteroaryl; wherein each alkyl, alkoxy, and cycloalkyl is unsubstituted or substituted with 1, 2, or 3 substituents selected from halogen, —OH, C₁₋₆ alkyl, and C₁₋₆ alkoxy;
n is 0-4; and
s is 1 or 2;
and
ii) a compound of Formula (B):

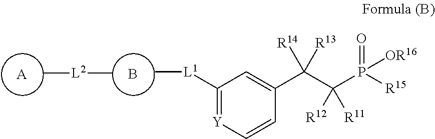

or a pharmaceutically acceptable salt, solvate, or stereoisomer thereof, wherein:
R¹¹, R¹², and R¹³ are each independently hydrogen, halogen, or C₁-C₆ alkyl;
R¹⁴ is hydrogen, C₁-C₆ alkyl, or C₃-C₆ cycloalkyl;
R¹⁵ is C₁-C₆ alkyl;
R¹⁶ is hydrogen or C₁-C₆ alkyl;
Y is CH or N;
L¹ is *—O—CH₂—, *—CH₂—O—, *—NR¹⁷—CH₂—, *—NR¹⁷—C(O)—, *—C(O)—NR¹⁷—, or *—C(O)—CH₂—;
   wherein * represents the connection to Ring B;
   R¹⁷ is hydrogen or C₁-C₆ alkyl;
Ring B is 3- to 6-membered heterocycloalkylene; wherein the heterocycloalkylene is unsubstituted or substituted with 1, 2, 3, or 4 R^{B} substituents;
or Ring B is C₃-C₆ cycloalkylene; wherein the cycloalkylene is unsubstituted or substituted with 1, 2, 3, or 4 R^{B} substituents;
   each R^{B} is independently halogen, C₁-C₆ alkyl, or C₁-C₆ fluoroalkyl;
L² is a bond or C₁-C₆ alkylene; wherein the alkylene is unsubstituted or substituted with 1, 2, or 3 substituents selected from the group consisting of —OH, C₁-C₆ alkyl, and —O—(C₁-C₆ alkyl);
Ring A is aryl or heteroaryl; wherein the aryl or heteroaryl is unsubstituted or substituted with 1, 2, or 3 R^{A} substituents;
each R^{A} is independently —F, —Cl, C₁-C₁₀ alkyl, C₁-C₁₀ fluoroalkyl, C₁-C₁₀ hydroxyalkyl, —OH, —OR¹⁸, —(C₁-C₆ alkylene)—OR¹⁸, —N(R¹⁹)₂, —(C₁-C₆ alkylene)-N(R¹⁹)₂;
each R¹⁸ is independently C₁-C₆ alkyl; wherein each alkyl is independently unsubstituted or substituted with 1, 2, 3, 4, or 5 substituents selected from the group consisting of halogen, —OH, C₁-C₆ alkyl and C₁-C₆ hydroxyalkyl; and
each R¹⁹ is independently hydrogen, C₁-C₆ alkyl, or monocyclic heteroaryl; wherein each alkyl and heteroaryl is independently unsubstituted or substituted with 1, 2, 3, 4, or 5 substituents selected from the group consisting of halogen, —OH, C₁-C₆ alkyl and C₁-C₆ hydroxyalkyl;
or two R¹⁹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

In some embodiments, the condition involving the gut-brain axis is a metabolic disorder. In some embodiments, the metabolic disorder is type 2 diabetes, hyperglycemia, metabolic syndrome, obesity, hypercholesterolemia, or nonalcoholic steatohepatitis. In some embodiments, the metabolic disorder is type 2 diabetes. In some embodiments, the metabolic disorder is obesity.

In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome.

In some embodiments, the condition involving the gut-brain axis is an eating disorder. In some embodiments, the eating disorder is hyperphagia, anorexia nervosa, or binge eating disorder. In some embodiments, the eating disorder is binge eating disorder.

Also disclosed herein is a method of weight management in an individual in need thereof comprising administering to the individual: i) a compound of Formula (A); and ii) a compound of Formula (B). In some embodiments, said weight management comprises one or more of: weight loss, maintenance of weight loss, decreased food consumption, increasing meal-related satiety, reducing pre-meal hunger, and reducing intra-meal food intake.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

This disclosure is directed, at least in part, to combination therapies useful for the treatment of conditions or disorders involving the gut-brain axis comprising a GPR119 agonist and a GPR40 agonist compound.

### Gut-Brain Axis

The gut-brain axis refers to the bidirectional biochemical signaling that connects the gastrointestinal tract (GI tract) with the central nervous system (CNS) through the peripheral nervous system (PNS) and endocrine, immune, and metabolic pathways.

In some instances, the gut-brain axis comprises the GI tract; the PNS including the dorsal root ganglia (DRG) and the sympathetic and parasympathetic arms of the autonomic nervous system including the enteric nervous system and the vagus nerve; the CNS; and the neuroendocrine and neuroimmune systems including the hypothalamic-pituitary-adrenal axis (HPA axis). The gut-brain axis is important for maintaining homeostasis of the body and is regulated and modulates physiology through the central and peripheral nervous systems and endocrine, immune, and metabolic pathways.

The gut-brain axis modulates several important aspects of physiology and behavior. Modulation by the gut-brain axis occurs via hormonal and neural circuits. Key components of these hormonal and neural circuits of the gut-brain axis include highly specialized, secretory intestinal cells that release hormones (enteroendocrine cells or EECs), the autonomic nervous system (including the vagus nerve and enteric nervous system), and the central nervous system. These systems work together in a highly coordinated fashion to modulate physiology and behavior.

Defects in the gut-brain axis are linked to a number of diseases, including those of high unmet need.

In some embodiments of the methods described herein, the condition or disorder involving the gut-brain axis is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, binge eating disorder, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis. In some embodiments, the condition is a metabolic disorder. In some embodiments, the metabolic disorder is type 2 diabetes, hyperglycemia, metabolic syndrome, obesity, hypercholesterolemia, or nonalcoholic steatohepatitis. In some embodiments, the metabolic disorder is diabetes. In other embodiments, the metabolic disorder is obesity. In other embodiments, the metabolic disorder is nonalcoholic steatohepatitis. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome. In some embodiments, the condition involving the gut-brain axis is weight loss or preventing weight gain or weight regain. In some embodiments, the condition involving the gut-brain axis is weight loss or preventing weight gain or weight regain post-bariatric surgery. In some embodiments, the condition involving the gut-brain axis is weight loss or preventing weight gain or weight regain, wherein the subject has had bariatric surgery.

### GPR119 in the Gut-Brain Axis

In some instances, GPR119 is expressed in the pancreas and in enteroendocrine cells of the gastrointestinal tract. In some instances, GPR119 is expressed in enteroendocrine cells. GPR119 is activated by oleoylethanolamide (OEA) and other oleic acid derivatives and N-acylethanolamides. GPR119 agonists may be useful in the treatment of metabolic diseases such as diabetes and obesity, and other diseases involving the gut-brain axis.

In some instances, modulators of GPR119, for example, GPR119 agonists, induce the production of intracellular cAMP. In some instances, modulators of GPR119, for example, GPR119 agonists, induce the secretion of GLP-1, GLP-2, GIP, PYY, CCK, or other hormones. In some instances, modulators of GPR119, for example, GPR119 agonists, induce the secretion of GLP-1, GIP, CCK or PYY. In some instances, modulators of GPR119, for example, GPR119 agonists, induce the secretion of GLP-1.

Described herein is a method of treating a condition or disorder involving the gut-brain axis in an individual in need thereof, the method comprising administering to the individual a GPR119 receptor modulator. In some embodiments, the GPR119 receptor modulator is a GPR119 agonist. In some embodiments, the GPR119 modulator is a gut-restricted GPR119 modulator.

### GPR40 in the Gut-Brain Axis

Free fatty acid receptor 1 (FFA1, FFAR1), also known as GPR40, is a class A G-protein coupled receptor. This membrane protein binds free fatty acids, acting as a nutrient sensor for regulating energy homeostasis. In some instances, GPR40 is expressed in enteroendocrine cells and pancreatic islet β cells. In some instances, GPR40 is expressed in enteroendocrine cells. Several naturally-occurring medium to long-chain fatty acids act as ligands for GPR40. GPR40 agonists or partial agonists may be useful in the treatment of metabolic diseases such as obesity, diabetes, and NASH, and other diseases involving the gut-brain axis.

In some instances, modulators of GPR40, for example, GPR40 agonists or partial agonists, induce insulin secretion. In some instances, modulators of GPR40, for example, GPR40 agonists or partial agonists, induce an increase in cytosolic Ca²⁺. In some instances, modulators of GPR40, for example, GPR40 agonists or partial agonists, induce higher levels of intracellular cAMP. In some instances, GPR40 modulation is in enteroendocrine cells. In some instances, modulators of GPR40, for example, GPR40 agonists, induce the secretion of GLP-1, GIP, CCK or PYY. In some instances, modulators of GPR40, for example, GPR40 agonists, induce the secretion of GLP-1.

Described herein is a method of treating a condition or disorder involving the gut-brain axis in an individual in need thereof, the method comprising administering to the individual a GPR40 receptor modulator. In some embodiments, the GPR40 receptor modulator is a GPR40 agonist or partial agonist. In some embodiments, the GPR40 receptor modulator is a GPR40 agonist. In some embodiments, the GPR40 receptor modulator is a GPR40 partial agonist. In some embodiments, the GPR40 receptor modulator is a GPR40 positive allosteric modulator. In some embodiments, the GPR40 modulator is a gut-restricted GPR40 modulator.

### Peptide Hormones of the Gut-Brain Axis

Incretins are a group of metabolic hormones released in the gut that stimulate a decrease in blood glucose levels in a glucose-dependent manner. Incretins include the peptide hormones GLP-1 and GIP. In some instances, incretin hormones are released in enteroendocrine cells after eating. In some instances, incretin hormones augment the secretion of insulin released from pancreatic beta cells of the islets of Langerhans by a blood glucose-dependent mechanism. In some instances, incretin hormones (such as GLP-1) also inhibit glucagon release from the alpha cells of the islets of Langerhans. Beside insulinotropic effects, GLP-1 has been associated with numerous regulatory and protective effects. GLP-1 inhibits gastric emptying, acid secretion, motility, decreases appetite and promotes satiety. GLP-1 receptor activation has been linked with neurotrophic effects including neurogenesis and neuroprotective effects including reduced necrotic and apoptotic signalling and cell death. GLP-1 receptor agonist treatment is associated with protection against a range of experimental disease models such as Parkinson's disease, Alzheimer's disease, stroke, traumatic brain injury, and multiple sclerosis. Other peptide hormones released in the gut include CCK, PYY, GLP-2, oxyntomdulin, gastrin, secretin, vasoactive intestinal peptide (VIP), motilin, ghrelin, bombesin, calcitonin gene-related peptide (CGRP), chromogranin A, enkephalins, enteroglucagon, galanin, ghrelin, growth factors, growth hormone-releasing factor, leptin, motilin, amylin, neuropeptide Y (NPY), neurotensin, pancreatic polypeptide, somatostatin, substance P and trefoil peptides. These peptides regulate a wide variety of processes including food intake, metabolic rate, glucose homeostasis, gastric emptying, gut motility, gall bladder contraction, pancreatic secretion, intestinal mucosal growth, muscosal protection and repair, pain, cell proliferation and differentiation, water and electrolyte secretion, and intestinal blood flow.

In some instances, modulating the activity of the GPCRs described herein, e.g., GPR40 and GPR119 increases peptide hormone secretion. In some instances, the biological effect of peptide hormones is in enteroendocrine cells. In some instances, peptide hormones, (e.g., GLP-1 and GIP), stimulate insulin release in a glucose dependent manner. In some instances, GLP-1, for example, is necessary for normal glucose homeostasis. In some instances, peptide hormones, (e.g., GLP-1, GLP-2 and GIP), contribute to beneficial effects for the treatment of diseases or conditions involving the gut-brain axis (e.g., diabetes, obesity or short bowel syndrome), including 1) increased insulin secretion, 2) increased glucose disposal, 3) suppression in glucose production, 4) reduced gastric emptying, 5) reduction in food intake, 6) body mass reduction, 7) increased cAMP levels, 8) increased nutrient absorption, 9) increased small intestinal length, 10) increased small intestinal weight, 11) increased villus height, and 12) increased villus height/crypt depth ratio.

### Combination Therapies

Described herein, in some embodiments, is a method of treating a condition or disorder involving the gut-brain axis in an individual in need thereof, the method comprising administering to the individual a GPR119 agonist and a GPR40 agonist. In some embodiments, the GPR119 agonist is a compound of Formula (A), as described herein. In some embodiments, the GPR40 agonist is a compound of Formula (B), as described herein.

In some embodiments, the condition or disorder is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, binge eating disorder, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis.

In some embodiments, the condition is a metabolic disorder. In some embodiments, the metabolic disorder is type 2 diabetes, hyperglycemia, metabolic syndrome, obesity, hypercholesterolemia, or nonalcoholic steatohepatitis. In some embodiments, the metabolic disorder is type 2 diabetes. In other embodiments, the metabolic disorder is obesity. In other embodiments, the metabolic disorder is nonalcoholic steatohepatitis.

In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome.

In some embodiments, the condition involving the gut-brain axis is an eating disorder. In some embodiments, the eating disorder is hyperphagia, anorexia nervosa, or binge eating disorder. In some embodiments, the eating disorder is binge eating disorder.

In some embodiments, the condition involving the gut-brain axis is weight loss or preventing weight gain or weight regain. In some embodiments, the condition involving the gut-brain axis is weight loss or preventing weight gain or weight regain post-bariatric surgery. In some embodiments, the condition involving the gut-brain axis is weight loss or preventing weight gain or weight regain, wherein the subject has had bariatric surgery.

Also described herein, in some embodiments, is a method of weight management in an individual in need thereof comprising administering to the individual a GPR119 agonist and a GPR40 agonist. In some embodiments, the GPR119 agonist is a compound of Formula (A), as described herein. In some embodiments, the GPR40 agonist is a compound of Formula (B), as described herein. In some embodiments, said weight management comprises one or more of: weight loss, maintenance of weight loss, decreased food consumption, increasing meal-related satiety, reducing pre-meal hunger, and reducing intra-meal food intake.

In some embodiments, modulating the activity of multiple receptors (e.g., GPR119 and GPR40) simultaneously as described herein results in elevated hormone levels. In some embodiments, modulating the activity of GPR119 and GPR40 simultaneously as described herein results in synergistically elevated hormone levels. In some embodiments, modulating the activity of GPR119 and GPR40 simultaneously as described herein results in elevated hormone secretion. In some embodiments, modulating the activity of GPR119 and GPR40 simultaneously as described herein results in synergistically elevated hormone secretion. In some embodiments, modulating the activity of GPR119 and GPR40 simultaneously as described herein results in hormone levels higher than those when modulating the activity of any single receptor. In some embodiments, modulating the activity of GPR119 and GPR40 simultaneously as described herein results in hormone secretion higher than that when modulating the activity of any single receptor. In some embodiments, modulating the activity of GPR119 and GPR40 simultaneously as described herein elicits a greater biological response, for example, increased insulin secretion, lower food consumption, increased body mass reduction, increased cAMP levels, increased nutrient absorption, increased small intestinal length, increased small intestinal weight, increased villus height, or increased villus height/crypt depth ratio than when modulating the activity of any single receptor. In some embodiments, modulating the activity of GPR119 and GPR40 simultaneously as described herein is preferred for the methods described herein relative to modulating the activity of a single receptor.

### Additional Therapeutic Agents

In certain embodiments, it is appropriate to administer the compound of Formula (A), or a pharmaceutically acceptable salt, solvate, or stereoisomer thereof, and the compound of Formula (B), or a pharmaceutically acceptable salt, solvate, or stereoisomer thereof, as described herein in combination with one or more additional therapeutic agents. In some embodiments, the combination therapies described herein are administered in combination with a TGR5 agonist, an SSTR5 antagonist, an SSTR5 inverse agonist, a CCK1 agonist, a PDE4 inhibitor, a DPP-4 inhibitor, a GLP-1 receptor agonist, a ghrelin O-acyltransferase (GOAT) inhibitor, metformin, or combinations thereof. In some embodiments, the combination therapies described herein are administered in combination with a DPP-4 inhibitor, a GLP-1 receptor agonist, metformin, or combinations thereof. In some embodiments, the combination therapies described herein are administered in combination with a DPP-4 inhibitor. In some embodiments, the combination therapies described herein are administered in combination with a GLP-1 receptor agonist. In some embodiments, the combination therapies described herein are administered in combination with metformin. In certain embodiments, the pharmaceutical composition further comprises one or more anti-diabetic agents. In certain embodiments, the pharmaceutical composition further comprises one or more anti-obesity agents. In certain embodiments, the pharmaceutical composition further comprises one or more agents to treat nutritional disorders.

### Compounds of the Disclosure

Described herein are combinations therapies for treating a condition or disorder involving the gut-brain axis in an individual in need thereof, the method comprising administering to the individual a GPR119 agonist and a GPR40 agonist. In some embodiments, the GPR119 agonist is a compound of Formula (A), as described herein. In some embodiments, the GPR40 agonist is a compound of Formula (B), as described herein.

### Compounds of Formula (A)

In some embodiments, described herein is a GPR119 agonist which is a compound of Formula (A):

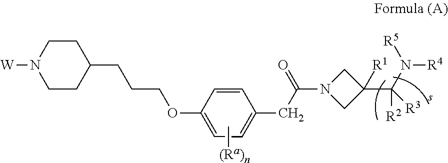

or a pharmaceutically acceptable salt, solvate, or stereoisomer thereof, wherein:
R¹ is hydrogen, —OH, or C₁₋₈ alkyl, wherein the alkyl is unsubstituted or substituted by —OH or —O(C₁₋₆ alkyl);
each R² and R³ is hydrogen;
or R² and R³ on the same carbon atom are taken together to form ═O;
R⁴ is hydrogen or C₁₋₆ alkyl;
R⁵ is C₁₋₈ alkyl, C₃-8 cycloalkyl, 4- to 8-membered heterocycloalkyl, —[(CH₂)ᵣ—Z]ₜ—R⁶, —[(CHR^{d})ᵣ—Z]ₜ—R⁶, or —[(C(R^{d})₂)ᵣ—Z]ₜ—R⁶; wherein each alkyl, cycloalkyl, and 4- to 8-membered heterocycloalkyl is substituted by 1-6 R^{c} groups each Z is independently —CH₂O—, —CH₂NR^{d}, —CH₂N⁺ (R^{d})₂—, or —NH—C(═O)—NH—;
   each r is independently 1-6;
   each t is independently 1-6;
   R⁶ is hydrogen, C₁₋₈ alkyl, C₃₋₈ cycloalkyl, or 4- to 8-membered heterocycloalkyl, wherein the alkyl, cycloalkyl, or 4- to 8-membered heterocycloalkyl is unsubstituted or substituted by 1-6 R^{c} groups;
or R⁴ and R⁵ are taken together with the nitrogen to which they are attached to form a 4- to 8-membered heterocycloalkyl, which is unsubstituted or substituted by 1-6 R^{c} groups;
each R^{c} is independently —OH, —CH₂OH, —NH₂, —N(R⁴)₃⁺, —C(═O)OH, —S(═O)₂OH, —S(═O)₂NH₂, —P(═O)(OH) 2, —P(═O)(OH)(R^{d}), —P(═O)(OH)(OR^{d}),

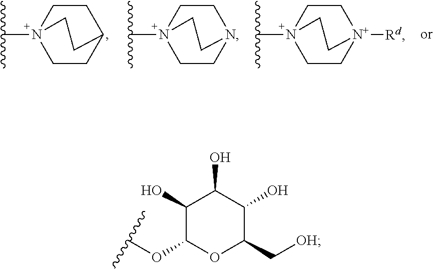

each R^{d} is independently C₁₋₆ alkyl;
each R^{a} is independently halogen, —CN, C₁₋₆ alkyl, C₁₋₆ fluoroalkyl, or C₃₋₆ cycloalkyl;
W is phenyl or 5-6 membered monocyclic heteroaryl, wherein the phenyl or heteroaryl is unsubstituted or substituted with 1, 2, or 3 substituents selected from R^{b};
   each R^{b} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from halogen, —OH, C₁₋₆ alkyl, and C₁₋₆ alkoxy;
n is 0-4; and
s is 1 or 2.

Compounds of Formula (A) are GPR119 agonists that are useful for the methods of treatment described herein. The preparation and uses of compounds of Formula (A) have been previously described (see, WO 2021/071837 and US 2022/0153719, each of which is incorporated by reference in its entirety).

Throughout the specification, groups and substituents thereof are chosen by one skilled in the field to provide stable moieties and compounds. Any combination of the groups described above or below for the various variables in Formula (A) is contemplated herein. For example, in some embodiments, W is phenyl or 6-membered monocyclic heteroaryl, wherein the heteroaryl is unsubstituted or substituted with 1 or 2 substituents selected from R^{b}. In some embodiments, W is 6-membered monocyclic heteroaryl, wherein the heteroaryl is unsubstituted or substituted with 1 or 2 substituents selected from R^{b}.

In some embodiments, each R^{b} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from halogen, —OH, C₁₋₆ alkyl, and C₁₋₆ alkoxy. In some embodiments, each R^{b} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, W is phenyl or 6-membered monocyclic heteroaryl, wherein the heteroaryl is unsubstituted or substituted with 1 or 2 substituents selected from R^{b}; and each R^{b} is independently halogen, —C(O)OH, —C(O)O(C₁₋₆ alkyl), C₁₋₆ alkyl, C₁₋₆ alkoxy, or C₃-6 cycloalkyl; wherein each alkyl, alkoxy, and cycloalkyl is unsubstituted or substituted with 1, 2, or 3 substituents selected from halogen, —OH, C₁₋₆ alkyl, and C₁₋₆ alkoxy. In some embodiments, W is 6-membered monocyclic heteroaryl, wherein the heteroaryl is unsubstituted or substituted with 1 or 2 substituents selected from R^{b}; and each R^{b} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, the compound of Formula (A) is a compound of Formula (A-I), or a pharmaceutically acceptable salt, solvate, or stereoisomer thereof:

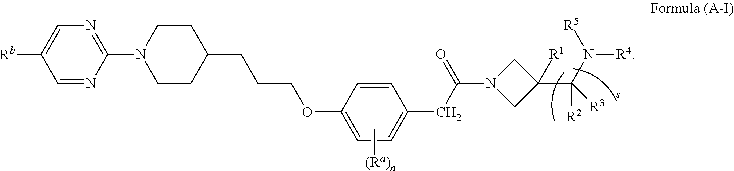

In some embodiments, R⁴ is hydrogen or C₁₋₄ alkyl. In some embodiments, R⁴ is hydrogen, methyl, ethyl, n-propyl, i-propyl, n-butyl, i-butyl, sec-butyl, or t-butyl. In some embodiments, R⁴ is hydrogen or methyl. In some embodiments, R⁴ is hydrogen.

In some embodiments, R⁵ is C₁₋₈ alkyl, —[(CH₂)ᵣ—Z]ₜ—R⁶, —[(CHR^{d})ᵣ—Z]-R⁶, or —[(C(R^{d})₂)ᵣ—Z]ₜ—R⁶; wherein the alkyl is substituted by 1-6 R^{c} groups; each Z is independently —CH₂O—, —CH₂NR^{d}, or —NH—C(═O)—NH—; r is 1-3; t is 1-3; and R⁶ is hydrogen or C₁₋₈ alkyl, wherein the alkyl is substituted by 1-6 R^{c} groups.

In some embodiments, R⁵ is C₁₋₈ alkyl which is substituted by 1-6 R^{c} groups.

In some embodiments, each R^{c} is independently —OH, —CH₂OH, —NH₂, —N(R^{d})₃⁺, —C(═O)OH, or

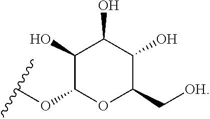 [SMILES: *O[C@H]1O[C@H](CO)[C@@H](O)[C@H](O)[C@@H]1O]

In some embodiments, each R^{c} is —OH.

In some embodiments, R⁵ is C₁₋₈ alkyl which is substituted by 1-6 R^{c} groups; and each R^{c} is independently —OH, —CH₂OH, —N(R⁴)₃⁺, —C(═O)OH, or

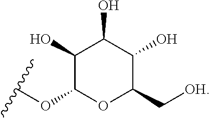 [SMILES: *O[C@H]1O[C@H](CO)[C@@H](O)[C@H](O)[C@@H]1O]

In some embodiments, R⁵ is C₁₋₈ alkyl which is substituted by 1-6 —OH groups.

In some embodiments, R⁴ is hydrogen or C₁₋₄ alkyl; R⁵ is C₁₋₈ alkyl, —[(CH₂)ᵣ—Z]ₜ—R⁶, —[(CHR^{d})ᵣ—Z]ₜ—R⁶, or —[(C(R^{d})₂)ᵣ—Z]ₜ—R⁶; wherein the alkyl is substituted by 1-6 R^{c} groups; each Z is independently —CH₂O—, —CH₂NR^{d}, or —NH—C(═O)—NH—; r is 1-3; t is 1-3; and R⁶ is hydrogen or C₁₋₈ alkyl, wherein the alkyl is substituted by 1-6 R^{c} groups.

In some embodiments, R⁴ is hydrogen or C₁₋₄ alkyl; R⁵ is C₁₋₈ alkyl which is substituted by 1-6 R^{c} groups; and each R^{c} is independently —OH, —CH₂OH, —N(R^{d})₃⁺, —C(═O)OH, or

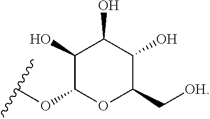 [SMILES: *O[C@H]1O[C@H](CO)[C@@H](O)[C@H](O)[C@@H]1O]

In some embodiments, R⁴ is hydrogen or methyl; R⁵ is C₁₋₈ alkyl which is substituted by 1-6 —OH groups.

In some embodiments, R⁴ and R⁵ are taken together with the nitrogen to which they are attached to form a 5- or 6-membered heterocycloalkyl, which is unsubstituted or substituted by 1-3 —OH groups.

In some embodiments, the compound of Formula (A) is a compound of Formula (A-II) or Formula (A-III), or a pharmaceutically acceptable salt, solvate, or stereoisomer thereof:

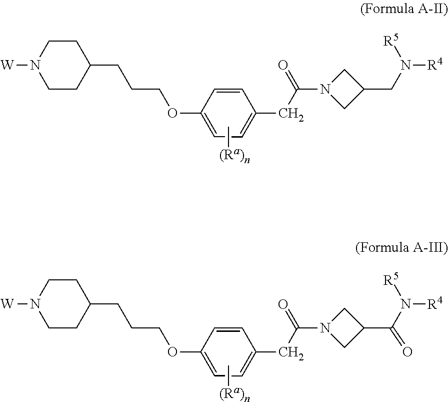

In some embodiments,
W is phenyl or 6-membered monocyclic heteroaryl, wherein the heteroaryl is unsubstituted or substituted with 1 or 2 substituents selected from R^{b};
each R^{b} is independently halogen, —C(O)OH, —C(O)O(C₁₋₆ alkyl), C₁₋₆ alkyl, C₁₋₆ alkoxy, or C₃-6 cycloalkyl; wherein each alkyl, alkoxy, and cycloalkyl is unsubstituted or substituted with 1, 2, or 3 substituents selected from halogen, —OH, C₁₋₆ alkyl, and C₁₋₆ alkoxy;
R⁴ is hydrogen or C₁₋₄ alkyl;
R⁵ is C₁₋₈ alkyl, —[(CH₂)ᵣ—Z]ₜ—R⁶, —[(CHR^{d})ₜ—Z]ₜ—R⁶, or —[(C(R^{d})₂)ᵣ—Z]ₜ—R⁶; wherein the alkyl is substituted by 1-6 R^{c} groups;
each Z is independently —CH₂O—, —CH₂NR^{d}, or —NH—C(═O)—NH—;
r is 1-3;
t is 1-3; and
R⁶ is hydrogen or C₁₋₈ alkyl, wherein the alkyl is substituted by 1-6 R^{c} groups.

In some embodiments,
W is 6-membered monocyclic heteroaryl, wherein the heteroaryl is unsubstituted or substituted with 1 or 2 substituents selected from R^{b};
each R^{b} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃;
R⁴ is hydrogen or C₁₋₄ alkyl;
R⁵ is C₁₋₈ alkyl which is substituted by 1-6 R^{c} groups; and each R^{c} is independently —OH, —CH₂OH, —N(R^{d})₃⁺, —C(═O)OH, or

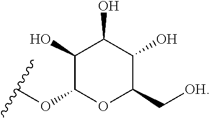 [SMILES: *O[C@H]1O[C@H](CO)[C@@H](O)[C@H](O)[C@@H]1O]

In some embodiments, the compound of Formula (A) that is useful for the methods described herein has a structure provided in Table 1.

TABLE 1

| Cpd # | Structure | Name |
|---|---|---|
| A1 | 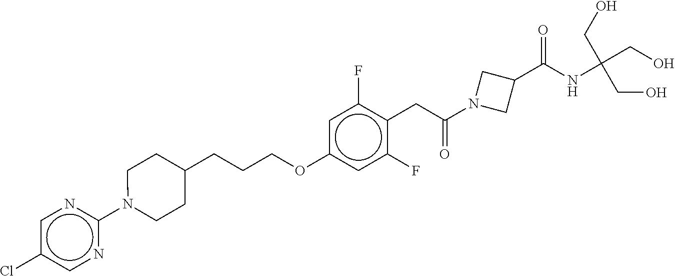 | 1-[2-[4-[3-[1-(5-chloropyrimidin-2-yl)-4- piperidyl]propoxy]-2,6-difluoro- phenyl]acetyl]-N-[2-hydroxy-1,1- bis(hydroxymethyl)ethyl]azetidine-3- carboxamide |
| A2 | 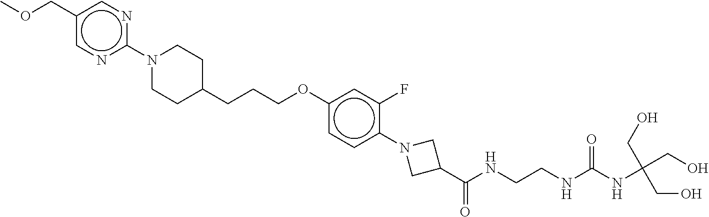 | 1-[2-[2-fluoro-4-[3-[1-[5- (methoxymethyl)pyrimidin-2-yl]-4- piperidyl]propoxy]phenyl]acetyl]-N-[2-[2- hydroxy-1,1- bis(hydroxymethyl)ethyl]carbamoylamino] ethyl]azetidine-3-carboxamide |
| A3 | 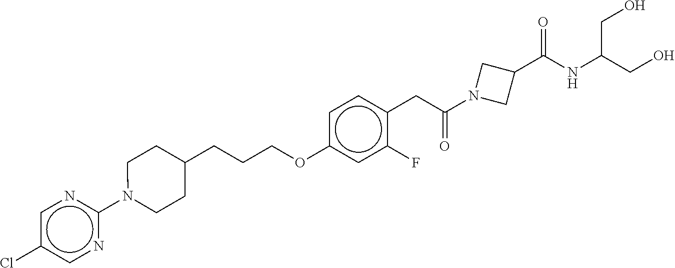 | 1-[2-[4-[3-[1-(5-chloropyrimidin-2-yl)-4- piperidyl]propoxy]-2-fluoro-phenyl]acetyl]- N-[2-hydroxy-1- (hydroxymethyl)ethyl]azetidine-3- carboxamide |
| A4 | 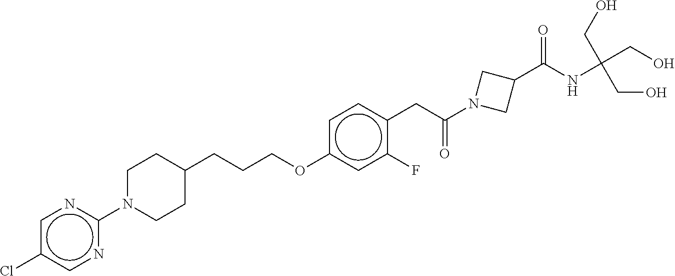 | 1-[2-[4-[3-[1-(5-chloropyrimidin-2-yl)-4- piperidyl]propoxy]-2-fluoro-phenyl]acetyl]- N-[2-hydroxy-1,1- bis(hydroxymethyl)ethyl]azetidine-3- carboxamide |
| A5 | 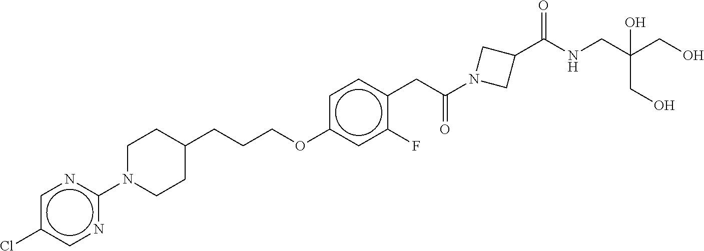 | 1-[2-[4-[3-[1-(5-chloropyrimidin-2-yl)-4- piperidyl]propoxy]-2-fluoro-phenyl]acetyl]- N-[2,3-dihydroxy-2- (hydroxymethyl)propyl]azetidine-3- carboxamide |
| A6 | 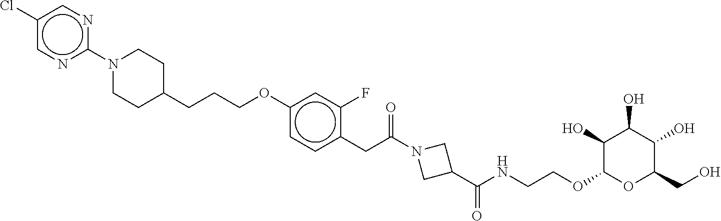 | 1-[2-[4-[3-[1-(5-chloropyrimidin-2-yl)-4- piperidyl]propoxy]-2-fluoro-phenyl]acetyl]- N-[2-[rac-(2S,3S,4S,5S,6R)-3,4,5- trihydroxy-6- (hydroxymethyl)tetrahydropyran-2- yl]oxyethyl]azetidine-3-carboxamide |
| A7 | 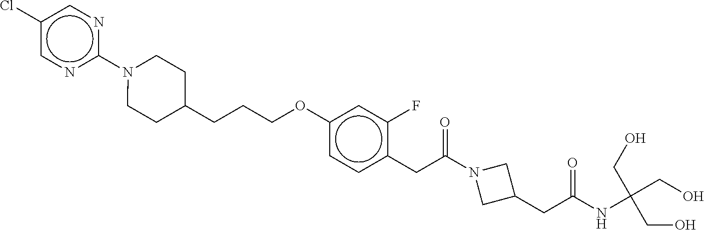 | 2-[1-[2-[4-[3-[1-(5-chloropyrimidin-2-yl)-4- piperidyl]propoxy]-2-fluoro- phenyl]acetyl]azetidin-3-yl]-N-[2-hydroxy- 1,1-bis(hydroxymethyl)ethyl]acetamide |
| A8 | 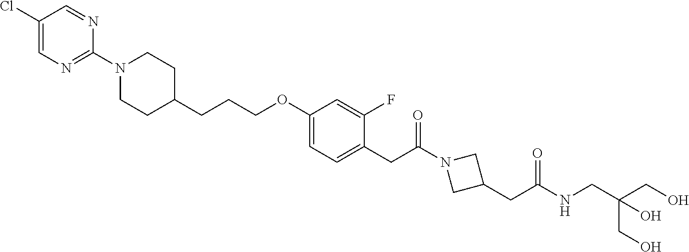 [SMILES: O=C(CC1CN(C(=O)Cc2ccc(OCCCC3CCN(c4ncc(Cl)cn4)CC3)cc2F)C1)NCC(O)(CO)CO] | 2-[1-[2-[4-[3-[1-(5-chloropyrimidin-2-yl)-4- piperidyl]propoxy]-2-fluoro- phenyl]acetyl]azetidin-3-yl]-N-[2,3- dihydroxy-2- (hydroxymethyl)propyl]acetamide |
| A9 | 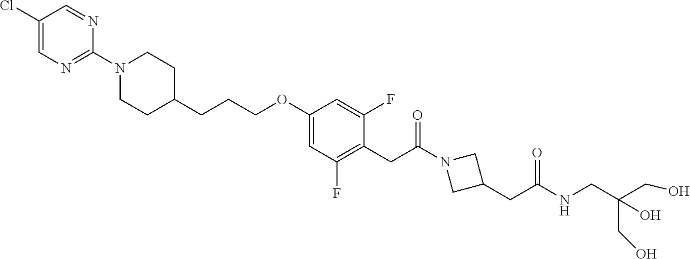 [SMILES: O=C(CC1CN(C(=O)Cc2c(F)cc(OCCCC3CCN(c4ncc(Cl)cn4)CC3)cc2F)C1)NCC(O)(CO)CO] | 2-[1-[2-[4-[3-[1-(5-chloropyrimidin-2-yl)-4- piperidyl]propoxy]-2,6-difluoro- phenyl]acetyl]azetidin-3-yl]-N-[2,3- dihydroxy-2- (hydroxymethyl)propyl]acetamide |
| A10 | 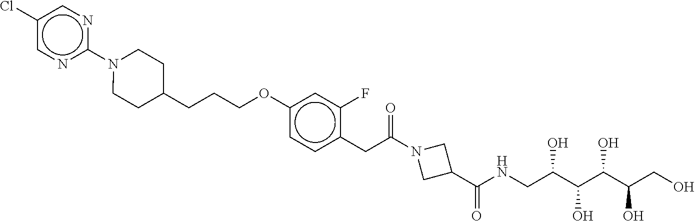 | 1-[2-[4-[3-[1-(5-chloropyrimidin-2-yl)-4- piperidyl]propoxy]-2-fluoro-phenyl]acetyl]- N-[rac-(2S,3R,4R,5R)-2,3,4,5,6- pentahydroxyhexyl]azetidine-3-carboxamide |
| A11 | 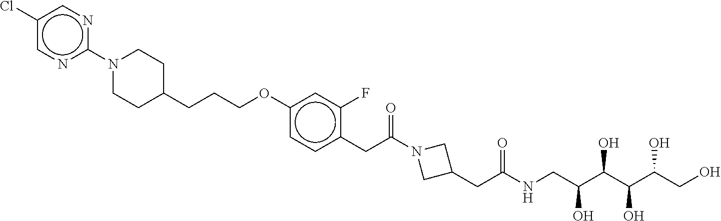 | 2-[1-[2-[4-[3-[1-(5-chloropyrimidin-2-yl)-4- piperidyl]propoxy]-2-fluoro- phenyl]acetyl]azetidin-3-yl]-N-[rac- (2S,3R,4R,5R)-2,3,4,5,6- pentahydroxyhexyl]acetamide |
| A12 | 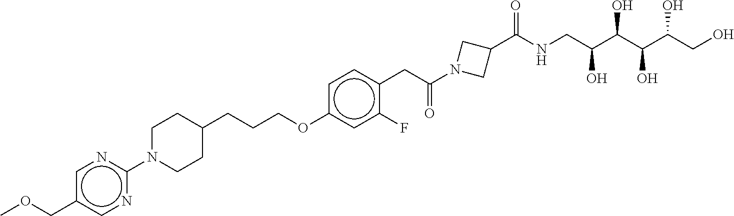 | 1-[2-[2-fluoro-4-[3-[1-[5- (methoxymethyl)pyrimidin-2-yl]-4- piperidyl]propoxy]phenyl]acetyl]-N-[rac- (2S,3R,4R,5R)-2,3,4,5,6- pentahydroxyhexyl]azetidine-3-carboxamide |
| A13 | 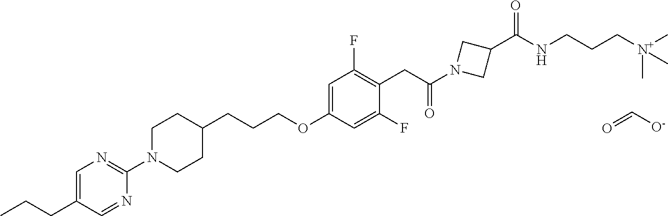 | 3-[[1-[2-[2,6-difluoro-4-[3-[1-(5- propylpyrimidin-2-yl)-4- piperidyl]propoxy]phenyl]acetyl]azetidine-3- carbonyl]amino]propyl-trimethyl-ammonium formate |
| A14 | 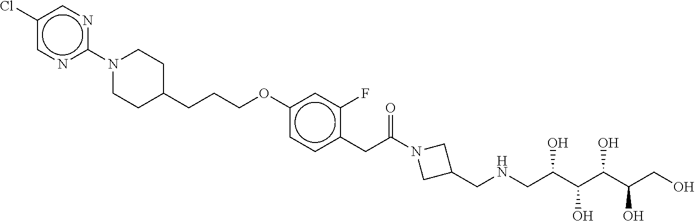 | 2-[4-[3-[1-(5-chloropyrimidin-2-yl)-4- piperidyl]propoxy]-2-fluoro-phenyl]-1-[3- [[[(2S,3R,4R,5R)-2,3,4,5,6- pentahydroxyhexyl]amino]methyl]azetidin- 1-yl]ethanone |
| A15 | 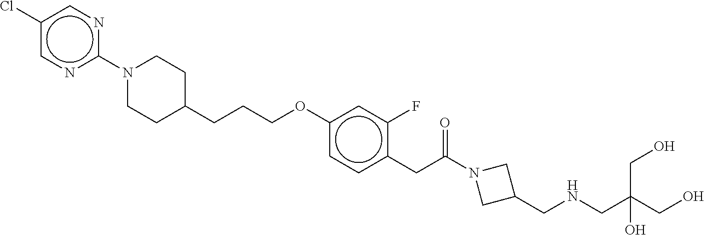 | 2-[4-[3-[1-(5-chloropyrimidin-2-yl)-4- piperidyl]propoxy]-2-fluoro-phenyl]-1-[3- [[[2,3-dihydroxy-2- (hydroxymethyl)propyl]amino]methyl] azetidin-1-yl]ethanone |
| A16 | 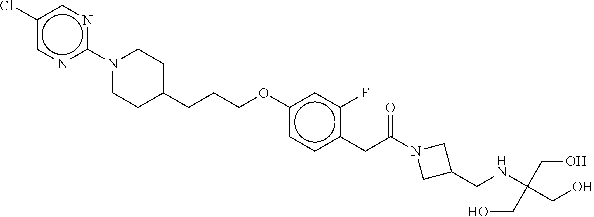 | 2-[4-[3-[1-(5-chloropyrimidin-2-yl)-4- piperidyl]propoxy]-2-fluoro-phenyl]-1-[3- [[[2-hydroxy-1,1- bis(hydroxymethyl)ethyl]amino]methyl] azetidin-1-yl]ethanone |
| A17 | 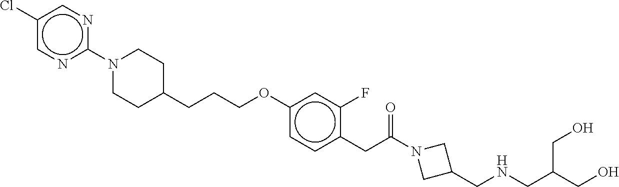 | 2-[4-[3-[1-(5-chloropyrimidin-2-yl)-4- piperidyl]propoxy]-2-fluoro-phenyl]-1-[3- [[[3-hydroxy-2- (hydroxymethyl)propyl]aminomethyl] azetidin-1-yl]ethanone |
| A18 | 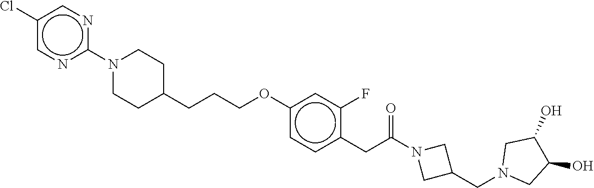 | 2-[4-[3-[1-(5-chloropyrimidin-2-yl)-4- piperidyl]propoxy]-2-fluoro-phenyl]-1-[3- [[rac-(3S,4S)-3,4-dihydroxypyrrolidin-1- yl]methyl]azetidin-1-yl]ethanone |
| A19 | 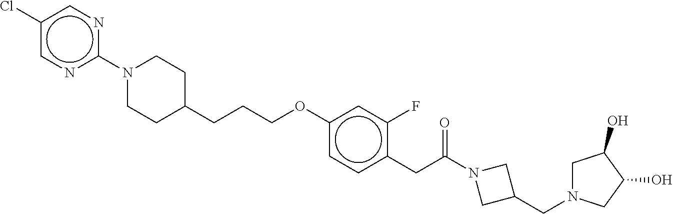 | 2-[4-[3-[1-(5-chloropyrimidin-2-yl)-4- piperidyl]propoxy]-2-fluoro-phenyl]-1-[3- [[rac-(3R,4R)-3,4-dihydroxypyrrolidin-1- yl]methyl]azetidin-1-yl]ethanone |
| A20 | 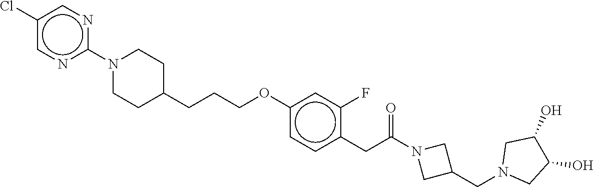 | 2-[4-[3-[1-(5-chloropyrimidin-2-yl)-4- piperidyl]propoxy]-2-fluoro-phenyl]-1-[3- [rac-(3S,4R)-3,4-dihydroxypyrrolidin-1- yl]methyl]azetidin-1-yl]ethanone |
| A21 | 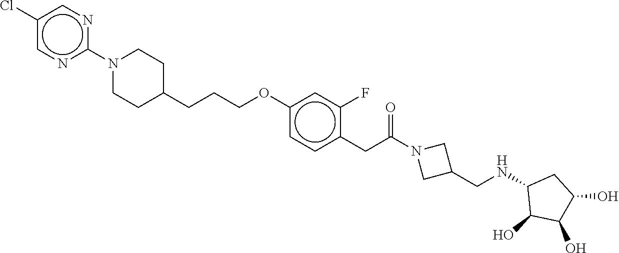 | 2-[4-[3-[1-(5-chloropyrimidin-2-yl)-4- piperidyl]propoxy]-2-fluoro-phenyl]-1-[3- [[[rac-(1R,2S,3R,4S)-2,3,4- trihydroxycyclopentyl]amino]methyl] azetidin-1-yl]ethanone |
| A22 | 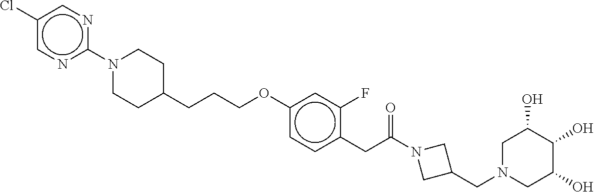 | 2-[4-[3-[1-(5-chloropyrimidin-2-yl)-4- piperidyl]propoxy]-2-fluoro-phenyl]-1-[3- [[rac-(3R,5S)-3,4,5-trihydroxy-1- piperidyl]methyl]azetidin-1-yl]ethanone |
| A23 | 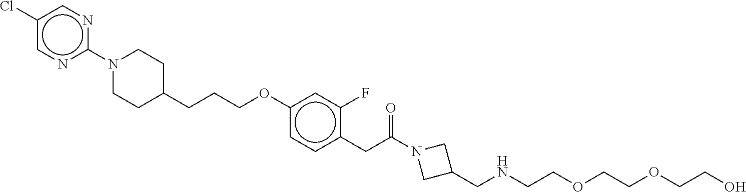 | 2-[4-[3-[1-(5-chloropyrimidin-2-yl)-4- piperidyl]propoxy]-2-fluoro-phenyl]-1-[3- [[2-[2-(2- hydroxyethoxy)ethoxy]ethylaminomethyl] azetidin-1-yl]ethanone |
| A24 | 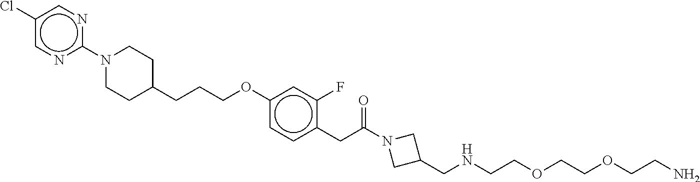 | 1-[3-[[2-[2-(2- aminoethoxy)ethoxy]ethylamino]methyl] azetidin-1-yl]-2-[4-[3-[1-(5-chloropyrimidin-2- yl)-4-piperidyl]propoxy]-2-fluoro- phenyl]ethanone |
| A25 | 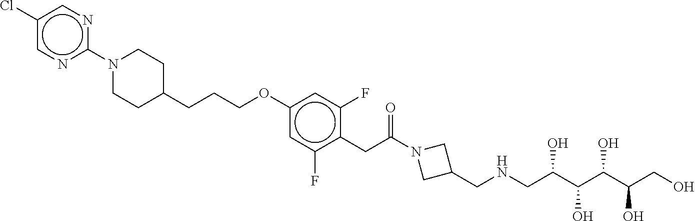 | 2-[4-[3-[1-(5-chloropyrimidin-2-yl)-4- piperidyl]propoxy]-2,6-difluoro-phenyl]-1- [3-[[[(2S,3R,4R,5R)-2,3,4,5,6- pentahydroxyhexyl]amino]methyl]azetidin- 1-yl]ethanone |
| A26 | 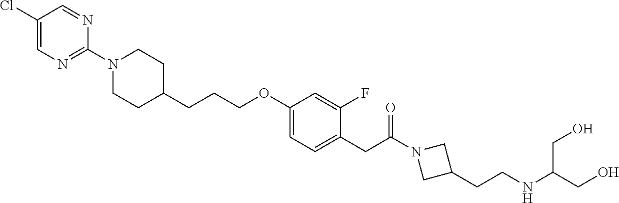 [SMILES: O=C(Cc1ccc(OCCCC2CCN(c3ncc(Cl)cn3)CC2)cc1F)N1CC(CCNC(CO)CO)C1] | 2-(4-(3-(1-(5-chloropyrimidin-2- yl)piperidin-4-yl)propoxy)-2-fluorophenyl)- 1-(3-(2-((1,3-dihydroxypropan-2- yl)amino)ethyl)azetidin-1-yl)ethan-1-one |
| A27 | 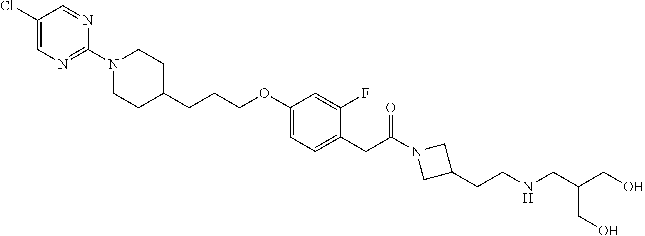 [SMILES: O=C(Cc1ccc(OCCCC2CCN(c3ncc(Cl)cn3)CC2)cc1F)N1CC(CCNCC(CO)CO)C1] | 2-(4-(3-(1-(5-chloropyrimidin-2- yl)piperidin-4-yl)propoxy)-2-fluorophenyl)- 1-(3-(2-((3-hydroxy-2- (hydroxymethyl)propyl)amino)ethyl)azetidin- 1-yl)ethan-1-one |
| A28 | 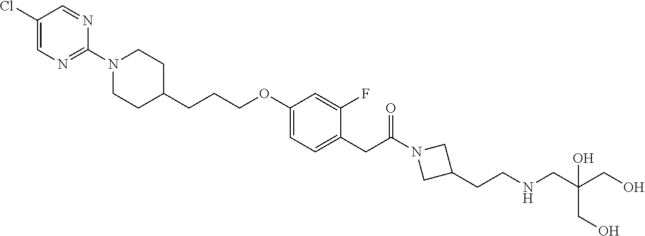 [SMILES: O=C(Cc1ccc(OCCCC2CCN(c3ncc(Cl)cn3)CC2)cc1F)N1CC(CCNCC(O)(CO)CO)C1] | 2-(4-(3-(1-(5-chloropyrimidin-2- yl)piperidin-4-yl)propoxy)-2-fluorophenyl)- 1-(3-(2-((2,3-dihydroxy-2- (hydroxymethyl)propyl)amino)ethyl)azetidin- 1-yl)ethan-1-one |
| A29 | 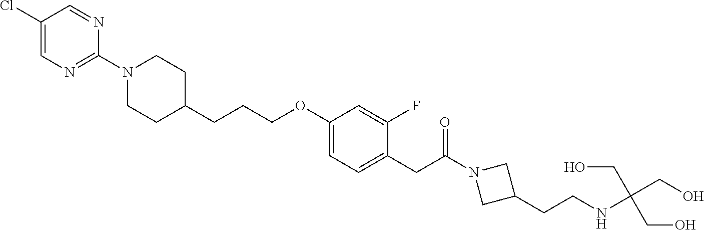 [SMILES: O=C(Cc1ccc(OCCCC2CCN(c3ncc(Cl)cn3)CC2)cc1F)N1CC(CCNC(CO)(CO)CO)C1] | 2-(4-(3-(1-(5-chloropyrimidin-2- yl)piperidin-4-yl)propoxy)-2-fluorophenyl)- 1-(3-(2-((1,3-dihydroxy-2- (hydroxymethyl)propan-2- yl)amino)ethyl)azetidin-1-yl)ethan-1-one |
| A30 | 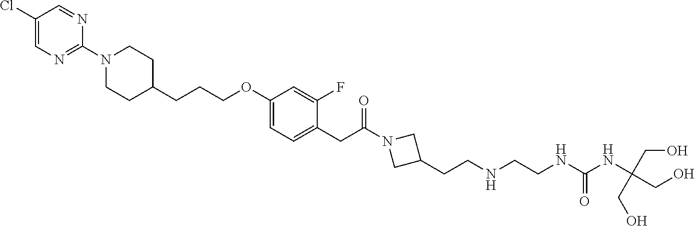 [SMILES: O=C(NCCNCCC1CN(C(=O)Cc2ccc(OCCCC3CCN(c4ncc(Cl)cn4)CC3)cc2F)C1)NC(CO)(CO)CO] | 1-(2-((2-(1-(2-(4-(3-(1-(5-chloropyrimidin-2- yl)piperidin-4-yl)propoxy)-2- fluorophenyl)acetyl)azetidin-3- yl)ethyl)amino)ethyl)-3-(1,3-dihydroxy-2- (hydroxymethyl)propan-2-yl)urea |
| A31 | 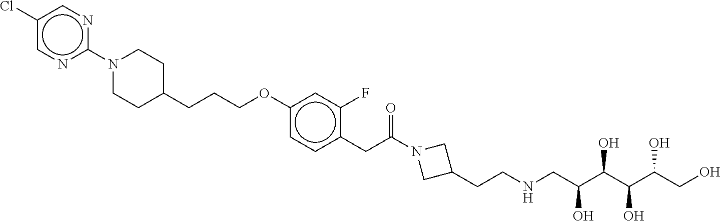 | 2-[4-[3-[1-(5-chloropyrimidin-2-yl)-4- piperidyl]propoxy]-2-fluoro-phenyl]-1-[3-[2- [[(2S,3R,4R,5R)-2,3,4,5,6- pentahydroxyhexyl]amino]ethyl]azetidin-1- yl]ethanone |
| A32 | 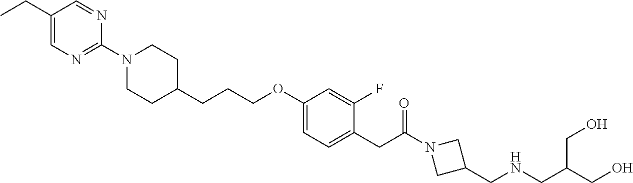 [SMILES: CCc1cnc(N2CCC(CCCOc3ccc(CC(=O)N4CC(CNCC(CO)CO)C4)c(F)c3)CC2)nc1] | 2-(4-(3-(1-(5-ethylpyrimidin-2-yl)piperidin- 4-yl)propoxy)-2-fluorophenyl)-1-(3-(((3- hydroxy-2- (hydroxymethyl)propyl)amino)methyl) azetidin-1-yl)ethan-1-one |
| A33 | 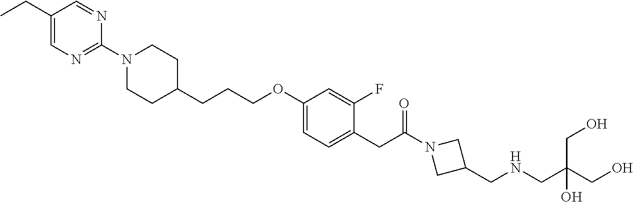 [SMILES: CCc1cnc(N2CCC(CCCOc3ccc(CC(=O)N4CC(CNCC(O)(CO)CO)C4)c(F)c3)CC2)nc1] | 1-(3-(((2,3-dihydroxy-2- (hydroxymethyl)propyl)amino)methyl) azetidin-1-yl)-2-(4-(3-(1-(5-ethylpyrimidin-2- yl)piperidin-4-yl)propoxy)-2- fluorophenyl)ethan-1-one |
| A34 | 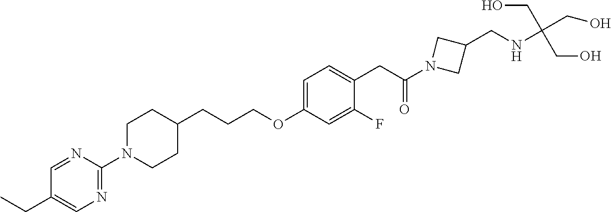 [SMILES: CCc1cnc(N2CCC(CCCOc3ccc(CC(=O)N4CC(CNC(CO)(CO)CO)C4)c(F)c3)CC2)nc1] | 1-(3-(((1,3-dihydroxy-2- (hydroxymethyl)propan-2- yl)amino)methyl)azetidin-1-yl)-2-(4-(3-(1- (5-ethylpyrimidin-2-yl)piperidin-4- yl)propoxy)-2-fluorophenyl)ethan-1-one |
| A35 | 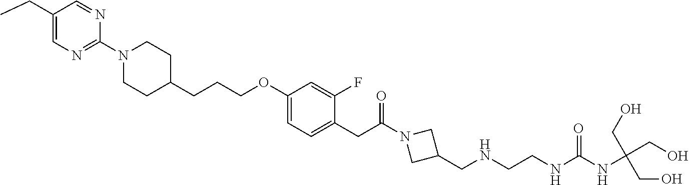 [SMILES: CCc1cnc(N2CCC(CCCOc3ccc(CC(=O)N4CC(CNCCNC(=O)NC(CO)(CO)CO)C4)c(F)c3)CC2)nc1] | 1-(1,3-dihydroxy-2-(hydroxymethyl)propan- 2-yl)-3-(2-(((1-(2-(4-(3-(1-(5- ethylpyrimidin-2-yl)piperidin-4-yl)propoxy)- 2-fluorophenyl)acetyl)azetidin-3- yl)methyl)amino)ethyl)urea |
| A36 | 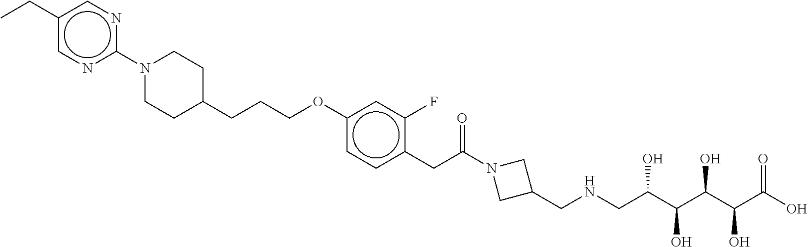 | (2S,3R,4S,5S)-6-[1-[2-[4-[3-[1-(5- ethylpyrimidin-2-yl)-4-piperidyl]propoxy]-2- fluoro-phenyl]acetyl]azetidin-3- yl]methylamino]-2,3,4,5-tetrahydroxy- hexanoic acid |
| A37 | 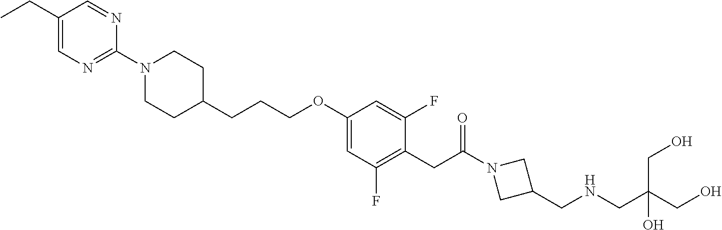 [SMILES: CCc1cnc(N2CCC(CCCOc3cc(F)c(CC(=O)N4CC(CNCC(O)(CO)CO)C4)c(F)c3)CC2)nc1] | 1-(3-(((2,3-dihydroxy-2- (hydroxymethyl)propyl)amino)methyl)azetidin- 1-yl)-2-(4-(3-(1-(5-ethylpyrimidin-2- yl)piperidin-4-yl)propoxy)-2,6- difluorophenyl)ethan-1-one |
| A38 | 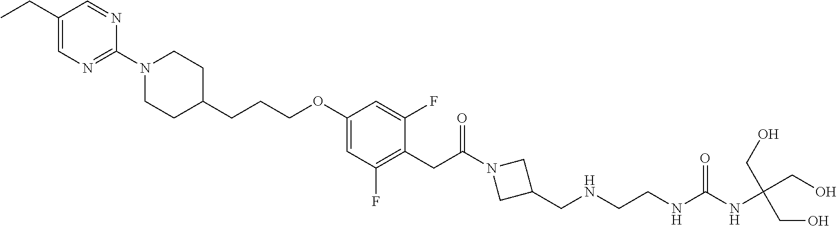 [SMILES: CCc1cnc(N2CCC(CCCOc3cc(F)c(CC(=O)N4CC(CNCCNC(=O)NC(CO)(CO)CO)C4)c(F)c3)CC2)nc1] | 1-(1,3-dihydroxy-2-(hydroxymethyl)propan- 2-yl)-3-(2-(((1-(2-(4-(3-(1-(5- ethylpyrimidin-2-yl)piperidin-4-yl)propoxy)- 2,6-difluorophenyl)acetyl)azetidin-3- yl)methyl)amino)ethyl)urea |
| A39 | 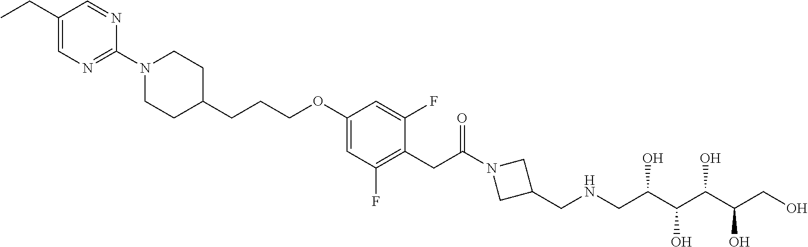 [SMILES: CCc1cnc(N2CCC(CCCOc3cc(F)c(CC(=O)N4CC(CNC[C@H](O)[C@@H](O)[C@H](O)[C@H](O)CO)C4)c(F)c3)CC2)nc1] | 2-(4-(3-(1-(5-ethylpyrimidin-2-yl)piperidin- 4-yl)propoxy)-2,6-difluorophenyl)-1-(3- ((((2S,3R,4R,5R)-2,3,4,5,6- pentahydroxyhexyl)amino)methyl)azetidin- 1-yl)ethan-1-one |
| A40 | 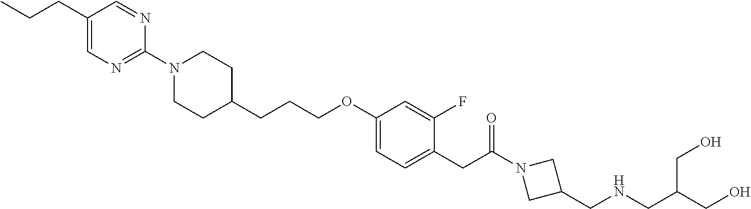 [SMILES: CCCc1cnc(N2CCC(CCCOc3ccc(CC(=O)N4CC(CNCC(CO)CO)C4)c(F)c3)CC2)nc1] | 2-(2-fluoro-4-(3-(1-(5-propylpyrimidin-2- yl)piperidin-4-yl)propoxy)phenyl)-1-(3-(((3- hydroxy-2- (hydroxymethyl)propyl)amino)methyl) azetidin-1-yl)ethan-1-one |
| A41 | 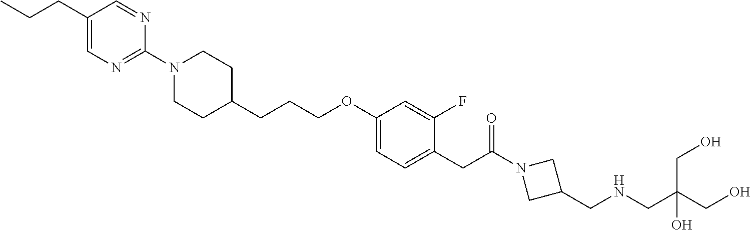 [SMILES: CCCc1cnc(N2CCC(CCCOc3ccc(CC(=O)N4CC(CNCC(O)(CO)CO)C4)c(F)c3)CC2)nc1] | 1-(3-(((2,3-dihydroxy-2- (hydroxymethyl)propyl)amino)methyl) azetidin-1-yl)-2-(2-fluoro-4-(3-(1-(5- propylpyrimidin-2-yl)piperidin-4- yl)propoxy)phenyl)ethan-1-one |
| A42 | 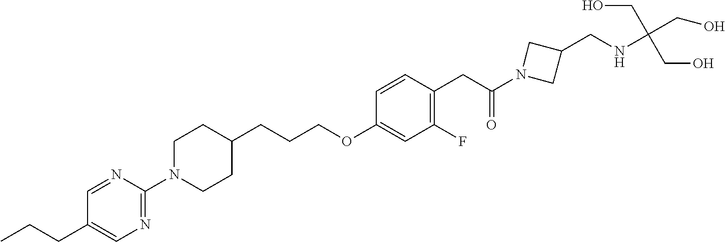 [SMILES: CCCc1cnc(N2CCC(CCCOc3ccc(CC(=O)N4CC(CNC(CO)(CO)CO)C4)c(F)c3)CC2)nc1] | 1-(3-(((1,3-dihydroxy-2- (hydroxymethyl)propan-2- yl)amino)methyl)azetidin-1-yl)-2-(2-fluoro- 4-(3-(1-(5-propylpyrimidin-2-yl)piperidin-4- yl)propoxy)phenyl)ethan-1-one |
| A43 | 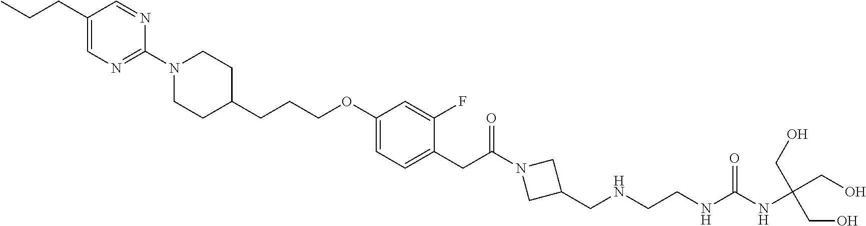 [SMILES: CCCc1cnc(N2CCC(CCCOc3ccc(CC(=O)N4CC(CNCCNC(=O)NC(CO)(CO)CO)C4)c(F)c3)CC2)nc1] | 1-(1,3-dihydroxy-2-(hydroxymethyl)propan- 2-yl)-3-(2-(((1-(2-(2-fluoro-4-(3-(1-(5- propylpyrimidin-2-yl)piperidin-4- yl)propoxy)phenyl)acetyl)azetidin-3- yl)methyl)amino)ethyl)urea |
| A44 | 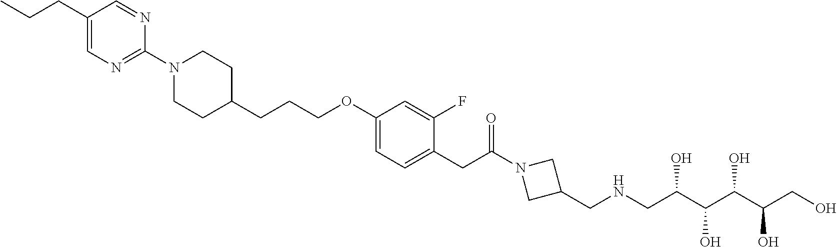 [SMILES: CCCc1cnc(N2CCC(CCCOc3ccc(CC(=O)N4CC(CNC[C@H](O)[C@@H](O)[C@H](O)[C@H](O)CO)C4)c(F)c3)CC2)nc1] | 2-(2-fluoro-4-(3-(1-(5-propylpyrimidin-2- yl)piperidin-4-yl)propoxy)phenyl)-1-(3- ((((2S,3R,4R,5R)-2,3,4,5,6- pentahydroxyhexyl)amino)methyl)azetidin- 1-yl)ethan-1-one |
| A45 | 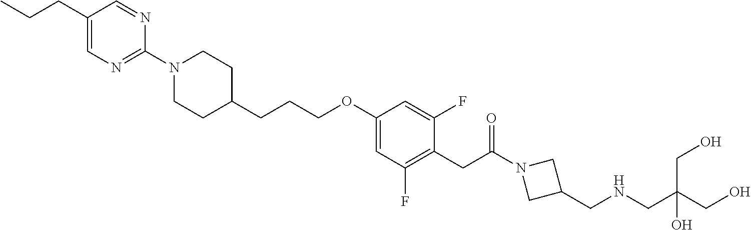 [SMILES: CCCc1cnc(N2CCC(CCCOc3cc(F)c(CC(=O)N4CC(CNCC(O)(CO)CO)C4)c(F)c3)CC2)nc1] | 2-(2,6-difluoro-4-(3-(1-(5-propylpyrimidin- 2-yl)piperidin-4-yl)propoxy)phenyl)-1-(3- (((2,3-dihydroxy-2- (hydroxymethyl)propyl)amino)methyl) azetidin-1-yl)ethan-1-one |
| A46 | 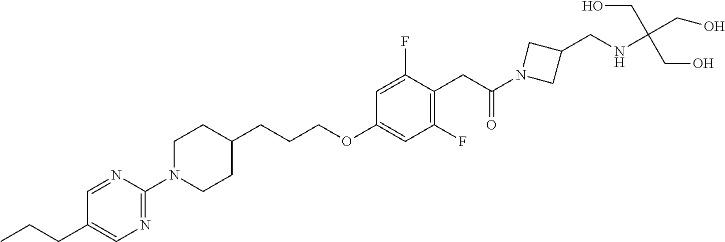 [SMILES: CCCc1cnc(N2CCC(CCCOc3cc(F)c(CC(=O)N4CC(CNC(CO)(CO)CO)C4)c(F)c3)CC2)nc1] | 2-(2,6-difluoro-4-(3-(1-(5-propylpyrimidin- 2-yl)piperidin-4-yl)propoxy)phenyl)-1-(3- (((1,3-dihydroxy-2-(hydroxymethyl)propan- 2-yl)amino)methyl)azetidin-1-yl)ethan-1-one |
| A47 | 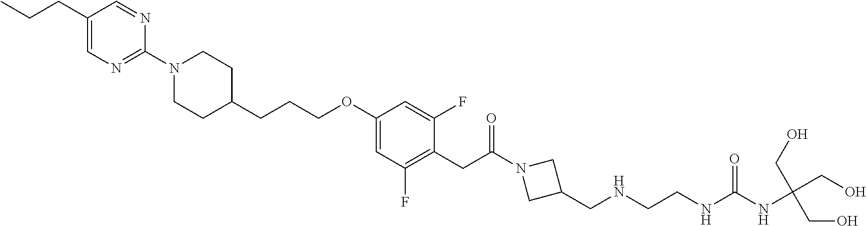 [SMILES: CCCc1cnc(N2CCC(CCCOc3cc(F)c(CC(=O)N4CC(CNCCNC(=O)NC(CO)(CO)CO)C4)c(F)c3)CC2)nc1] | 1-(2-(((1-(2-(2,6-difluoro-4-(3-(1-(5- propylpyrimidin-2-yl)piperidin-4- yl)propoxy)phenyl)acetyl)azetidin-3- yl)methyl)amino)ethyl)-3-(1,3-dihydroxy-2- (hydroxymethyl)propan-2-yl)urea |
| A48 | 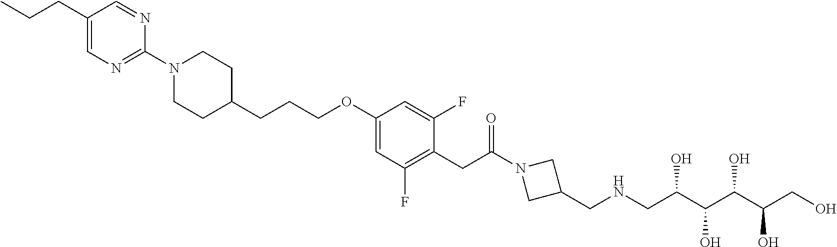 [SMILES: CCCc1cnc(N2CCC(CCCOc3cc(F)c(CC(=O)N4CC(CNC[C@H](O)[C@@H](O)[C@H](O)[C@H](O)CO)C4)c(F)c3)CC2)nc1] | 2-(2,6-difluoro-4-(3-(1-(5-propylpyrimidin- 2-yl)piperidin-4-yl)propoxy)phenyl)-1-(3- ((((2S,3R,4R,5R)-2,3,4,5,6- pentahydroxyhexyl)amino)methyl)azetidin- 1-yl)ethan-1-one |
| A49 | 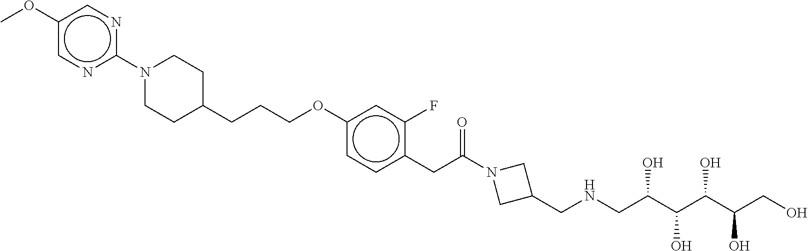 | 2-[2-fluoro-4-[3-[1-(5-methoxypyrimidin-2- yl)-4-piperidyl]propoxy]phenyl]-1-[3-[rac- (2S,3R,4R,5R)-2,3,4,5,6- pentahydroxyhexyl]amino]methyl]azetidin- 1-yl]ethanone |
| A50 | 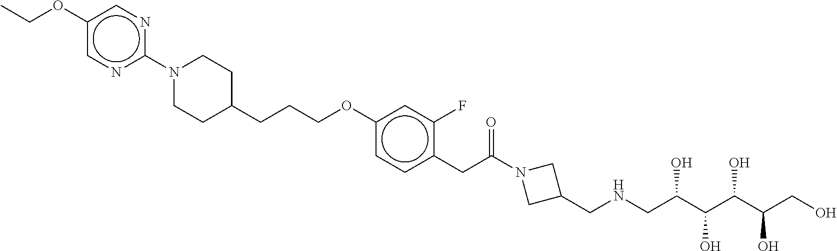 | 2-[4-[3-[1-(5-ethoxypyrimidin-2-yl)-4- piperidyl]propoxy]-2-fluoro-phenyl]-1-[3- [[[rac-(2S,3R,4R,5R)-2,3,4,5,6- pentahydroxyhexyl]amino]methyl]azetidin- 1-yl]ethanone |
| A51 | 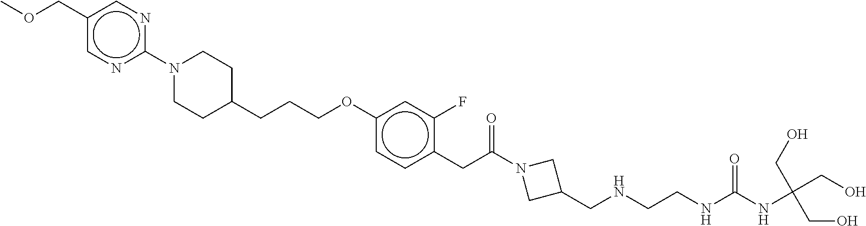 | 1-[2-[1-[2-[2-fluoro-4-[3-[1-[5- (methoxymethyl)pyrimidin-2-yl]-4- piperidyl]propoxy]phenyl]acetyl]azetidin-3- yl]methylamino]ethyl]-3-[2-hydroxy-1,1- bis(hydroxymethyl)ethyl]urea |
| A52 | 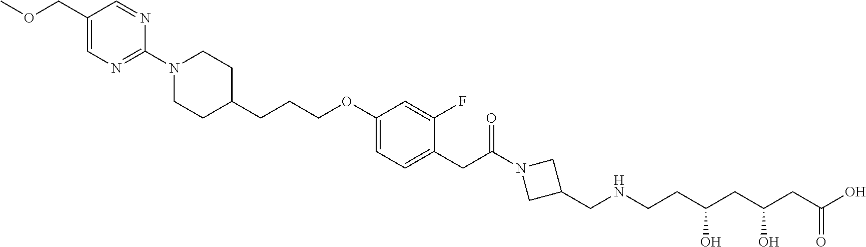 [SMILES: COCc1cnc(N2CCC(CCCOc3ccc(CC(=O)N4CC(CNCC[C@@H](O)C[C@@H](O)CC(=O)O)C4)c(F)c3)CC2)nc1] | (3R,5R)-7-[[1-[2-[2-fluoro-4-[3-[1-[5- (methoxymethyl)pyrimidin-2-yl]-4- piperidyl]propoxy]phenyl]acetyl]azetidin-3- yl]methylamino]-3,5-dihydroxy-heptanoic acid |
| A53 | 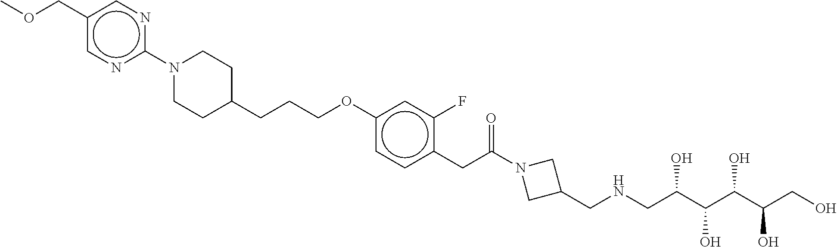 | 2-[2-fluoro-4-[3-[1-[5- (methoxymethyl)pyrimidin-2-yl]-4- piperidyl]propoxy]phenyl]-1-[3- [[[(2S,3R,4R,5R)-2,3,4,5,6- pentahydroxyhexyl]amino]methyl]azetidin- 1-yl]ethanone |
| A54 | 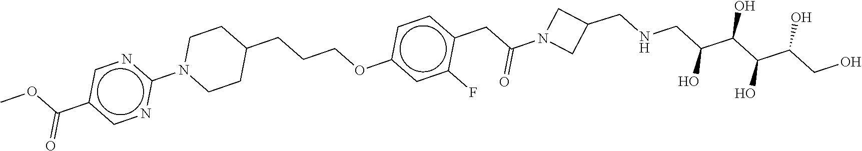 | methyl 2-[4-[3-[3-fluoro-4-[2-oxo-2-[3- [[[(2S,3R,4R,5R)-2,3,4,5,6- pentahydroxyhexyl]amino]methyl]azetidin- 1-yl]ethyl]phenoxy]propyl]-1- piperidyl]pyrimidine-5-carboxylate |
| A55 | 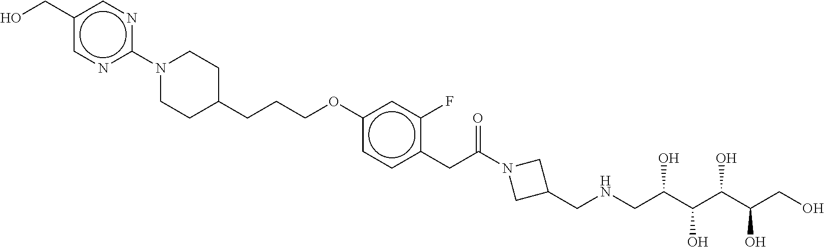 | 2-[2-fluoro-4-[3-[1-[5- (hydroxymethyl)pyrimidin-2-yl]-4- piperidyl]propoxy]phenyl]-1-[3- [[[(2S,3R,4R,5R)-2,3,4,5,6- pentahydroxyhexyl]amino]methyl]azetidin- 1-yl]ethanone |
| A56 | 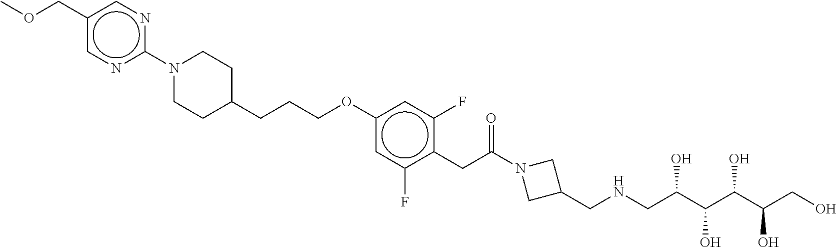 | 2-[2,6-difluoro-4-[3-[1-[5- (methoxymethyl)pyrimidin-2-yl]-4- piperidyl]propoxy]phenyl]-1-[3- [[[(2S,3R,4R,5R)-2,3,4,5,6- pentahydroxyhexyl]aminomethyl]azetidin- 1-yl]ethanone |
| A57 | 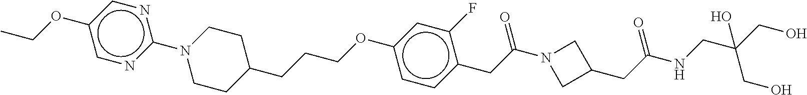 | N-[2,3-dihydroxy-2- (hydroxymethyl)propyl]-2-[1-[2-[4-[3-[1-(5- ethoxypyrimidin-2-yl)-4-piperidyl]propoxy]- 2-fluoro-phenyl]acetyl]azetidin-3- yl]acetamide |
| A58 | 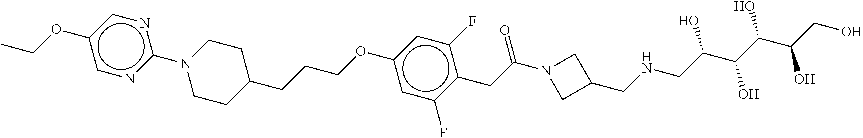 | 2-[4-[3-[1-(5-ethoxypyrimidin-2-yl)-4- piperidyl]propoxy]-2,6-difluoro-phenyl]-1- [3-[[[(2S,3R,4R,5R)-2,3,4,5,6- pentahydroxyhexyl]amino]methyl]azetidin- 1-yl]ethanone |
| A59 | 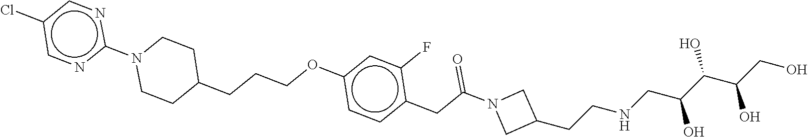 | 2-[4-[3-[1-(5-chloropyrimidin-2-yl)-4- piperidyl]propoxy]-2-fluoro-phenyl]-1-[3-[2- [[(2S,3S,4R)-2,3,4,5- tetrahydroxypentyl]amino]ethyl]azetidin-1- yl]ethanone |
| A60 | 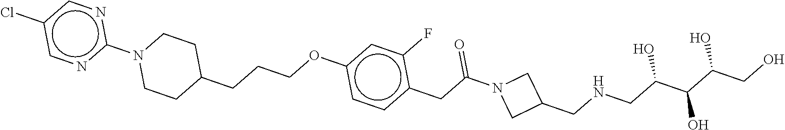 | 2-[4-[3-[1-(5-chloropyrimidin-2-yl)-4- piperidyl]propoxy]-2-fluoro-phenyl]-1-[3- [[[(2S,3S,4R)-2,3,4,5- tetrahydroxypentyl]amino]methyl]azetidin- 1-yl]ethanone |
| A61 | 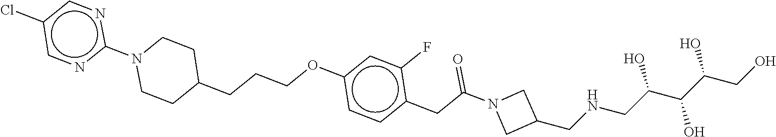 | 2-[4-[3-[1-(5-chloropyrimidin-2-yl)-4- piperidyl]propoxy]-2-fluoro-phenyl]-1-[3- [[[(2S,3R,4R)-2,3,4,5- tetrahydroxypentyl]amino]methyl]azetidin- 1-yl]ethanone |
| A62 | 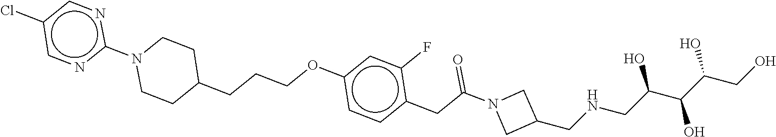 | 2-[4-[3-[1-(5-chloropyrimidin-2-yl)-4- piperidyl]propoxy]-2-fluoro-phenyl]-1-[3- [[[(2R,3S,4R)-2,3,4,5- tetrahydroxypentyl]amino]methyl]azetidin- 1-yl]ethanone |
| A63 | 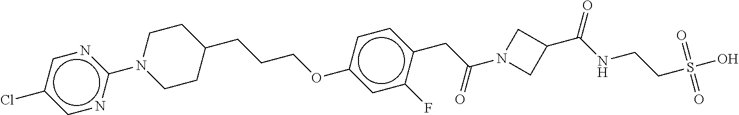 | 2-[[1-[2-[4-[3-[1-(5-chloropyrimidin-2-yl)-4- piperidyl]propoxy]-2-fluoro- phenyl]acetyl]azetidine-3- carbonyl]amino]ethanesulfonic acid |
| A64 | 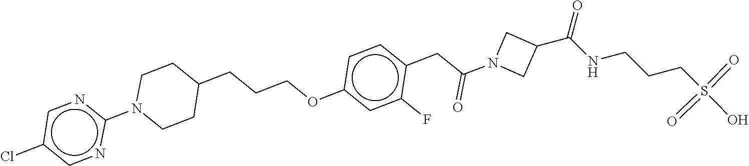 | 3-[[1-[2-[4-[3-[1-(5-chloropyrimidin-2-yl)-4- piperidyl]propoxy]-2-fluoro- phenyl]acetyl]azetidine-3- carbonyl]amino]propane-1-sulfonic acid |
| A65 | 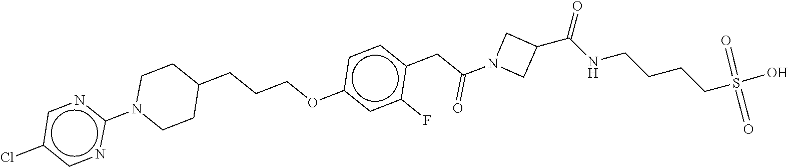 | 4-[[1-[2-[4-[3-[1-(5-chloropyrimidin-2-yl)-4- piperidyl]propoxy]-2-fluoro- phenyl]acetyl]azetidine-3- carbonyl]amino]butane-1-sulfonic acid |
| A66 | 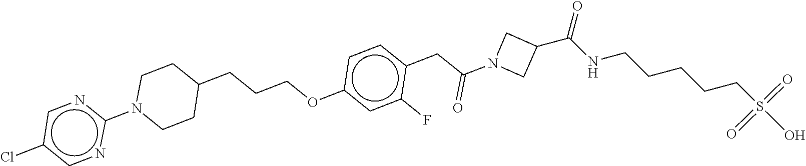 | 5-[[1-[2-[4-[3-[1-(5-chloropyrimidin-2-yl)-4- piperidyl]propoxy]-2-fluoro- phenyl]acetyl]azetidine-3- carbonyl]amino]pentane-1-sulfonic acid |
| A67 | 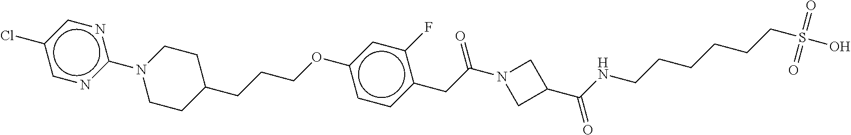 | 6-[[1-[2-[4-[3-[1-(5-chloropyrimidin-2-yl)-4- piperidyl]propoxy]-2-fluoro- phenyl]acetyl]azetidine-3- carbonyl]amino]hexane-1-sulfonic acid |
| A68 | 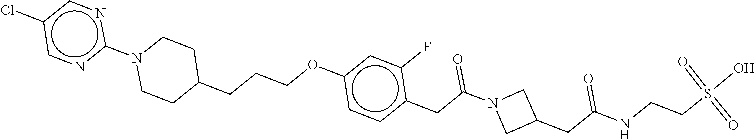 | 2-[[2-[1-[2-[4-[3-[1-(5-chloropyrimidin-2- yl)-4-piperidyl]propoxy]-2-fluoro- phenyl]acetyl]azetidin-3- yl]acetyl]amino]ethanesulfonic acid |
| A69 | 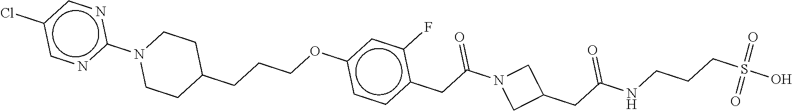 | 3-[[2-[1-[2-[4-[3-[1-(5-chloropyrimidin-2- yl)-4-piperidyl]propoxy]-2-fluoro- phenyl]acetyl]azetidin-3- yl]acetyl]amino]propane-1-sulfonic acid |
| A70 | 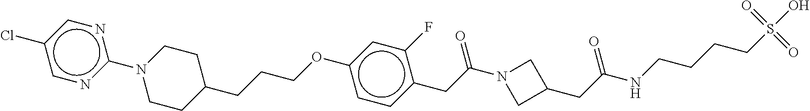 | 4-[[2-[1-[2-[4-[3-[1-(5-chloropyrimidin-2- yl)-4-piperidyl]propoxy]-2-fluoro- phenyl]acetyl]azetidin-3- yl]acetyl]amino]butane-1-sulfonic acid |
| A71 | 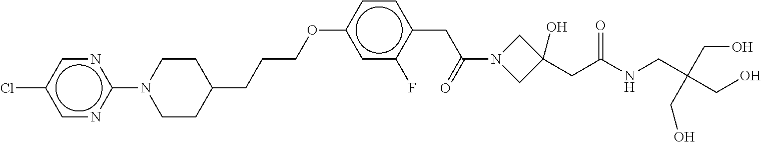 | 2-[1-[2-[4-[3-[1-(5-chloropyrimidin-2-yl)-4- piperidyl]propoxy]-2-fluoro-phenyl]acetyl]- 3-hydroxy-azetidin-3-yl]-N-[3-hydroxy-2,2- bis(hydroxymethyl)propyl]acetamide |
| A72 | 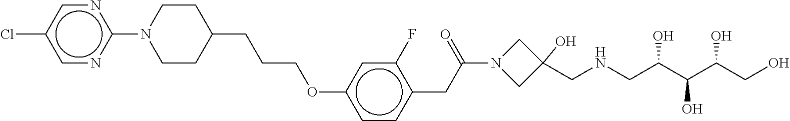 | 2-[4-[3-[1-(5-chloropyrimidin-2-yl)-4- piperidyl]propoxy]-2-fluoro-phenyl]-1-[3- hydroxy-3-[[[(2S,3S,4R)-2,3,4,5- tetrahydroxypentyl]amino]methyl]azetidin- 1-yl]ethanone |

In some embodiments, the compound of Formula (A) that is useful for the methods described herein is a pharmaceutically acceptable salt of a compound in Table 1.

### Compounds of Formula (B)

In some embodiments, described herein is a GPR40 agonist which is a compound of Formula (B):

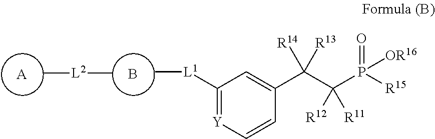

or a pharmaceutically acceptable salt, solvate, or stereoisomer thereof, wherein:
R¹¹, R¹², and R¹³ are each independently hydrogen, halogen, or C₁-C₆ alkyl;
R¹⁴ is hydrogen, C₁-C₆ alkyl, or C₃-C₆ cycloalkyl;
R¹⁵ is C₁-C₆ alkyl;
R¹⁶ is hydrogen or C₁-C₆ alkyl;
Y is CH or N;
L¹ is *—O—CH₂—, *—CH₂—O—, *—NR¹⁷—CH₂—, *—NR¹⁷—C(O)—, *—C(O)—NR¹⁷—, or *—C(O)—CH₂—;
   wherein * represents the connection to Ring B;
   R¹⁷ is hydrogen or C₁-C₆ alkyl;
Ring B is 3- to 6-membered heterocycloalkylene; wherein the heterocycloalkylene is unsubstituted or substituted with 1, 2, 3, or 4 R^{B} substituents;
or Ring B is C₃-C₆ cycloalkylene; wherein the cycloalkylene is unsubstituted or substituted with 1, 2, 3, or 4 R^{B} substituents;
   each R^{B} is independently halogen, C₁-C₆ alkyl, or C₁-C₆ fluoroalkyl;
L² is a bond or C₁-C₆ alkylene; wherein the alkylene is unsubstituted or substituted with 1, 2, or 3 substituents selected from the group consisting of —OH, C₁-C₆ alkyl, and —O—(C₁-C₆ alkyl);
Ring A is aryl or heteroaryl; wherein the aryl or heteroaryl is unsubstituted or substituted with 1, 2, or 3 R^{A} substituents;
each R^{A} is independently —F, —Cl, C₁-C₁₀ alkyl, C₁-C₁₀ fluoroalkyl, C₁-C₁₀ hydroxyalkyl, —OH, —OR¹⁸, —(C₁-C₆ alkylene)—OR¹⁸, —N(R¹⁹)₂, —(C₁-C₆ alkylene)-N(R¹⁹)₂;
each R¹⁸ is independently C₁-C₆ alkyl; wherein each alkyl is independently unsubstituted or substituted with 1, 2, 3, 4, or 5 substituents selected from the group consisting of halogen, —OH, C₁-C₆ alkyl and C₁-C₆ hydroxyalkyl; and
each R¹⁹ is independently hydrogen, C₁-C₆ alkyl, or monocyclic heteroaryl; wherein each alkyl and heteroaryl is independently unsubstituted or substituted with 1, 2, 3, 4, or 5 substituents selected from the group consisting of halogen, —OH, C₁-C₆ alkyl and C₁-C₆ hydroxyalkyl;
or two R¹⁹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

Compounds of Formula (B) are GPR40 agonists that are useful for the methods of treatment described herein. The preparation and uses of compounds of Formula (B) have been previously described (see, WO 2021/174048 and U.S. application Ser. No. 17/745,126, each of which is incorporated by reference in its entirety).

Throughout the specification, groups and substituents thereof are chosen by one skilled in the field to provide stable moieties and compounds. Any combination of the groups described above or below for the various variables in Formula (B) is contemplated herein. For example, in some embodiments, Y is CH. In other embodiments, Y is N.

In some embodiments, R¹¹, R¹², and R¹³ are each independently hydrogen, halogen, or C₁-C₄ alkyl. In some embodiments, R¹¹, R¹², and R¹³ are each independently hydrogen or C₁-C₄ alkyl. In some embodiments, R¹¹, R¹², and R¹³ are each independently hydrogen, methyl, ethyl, n-propyl, i-propyl, n-butyl, i-butyl, sec-butyl, or t-butyl. In some embodiments, R¹¹, R¹², and R¹³ are each independently hydrogen, methyl, or ethyl.

In some embodiments, R¹⁴ is hydrogen. In other embodiments, R 14 is C₁-C₆ alkyl or C₃-C₆ cycloalkyl. In some embodiments, R¹⁴ is methyl, ethyl, n-propyl, i-propyl, n-butyl, i-butyl, sec-butyl, t-butyl, or cyclopropyl. In some embodiments, R¹⁴ is hydrogen, methyl, ethyl, or cyclopropyl. In some embodiments, R¹⁴ is unsubstituted C₃-C₆ cycloalkyl. In some embodiments, R¹⁴ is cyclopropyl.

In some embodiments, R¹¹, R¹², and R¹³ are each independently hydrogen, halogen, or C₁-C₄ alkyl; and R¹⁴ is unsubstituted C₃-C₆ cycloalkyl.

In some embodiments, the compound of Formula (B) is a compound of Formula (B-I):

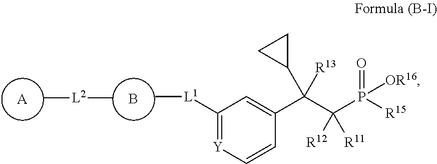

or a pharmaceutically acceptable salt, solvate, or stereoisomer thereof. In some embodiments,
R¹, R², and R³ are each independently hydrogen, —F, —Cl, or C₁-C₄ alkyl.

In some embodiments, L¹ is *—O—CH₂- or *—CH₂—O—; wherein * represents the connection to Ring B. In some embodiments, L¹ is *—O—CH₂—; wherein * represents the connection to Ring B. In some embodiments, L¹ is *—CH₂—O—; wherein * represents the connection to Ring B.

In some embodiments, Ring B is 3- to 6-membered heterocycloalkylene; wherein the heterocycloalkylene is unsubstituted or substituted with 1, 2, 3, or 4 R^{B} substituents.

In some embodiments, the compound of Formula (B) is a compound of Formula (B-II):

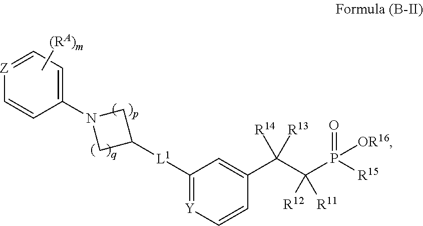

or a pharmaceutically acceptable salt, solvate, or stereoisomer thereof; wherein Z is N, CH, or CR^{A}; each R^{A} is independently —F, —Cl, C₁-C₇ alkyl, C₁-C₄ fluoroalkyl, —OH, or —OR¹⁰; p and q are each independently 1 or 2; and m is 0, 1, or 2.

In some embodiments, the compound of Formula (B-II) is a compound of Formula (B-III), or a pharmaceutically acceptable salt, solvate, or stereoisomer thereof:

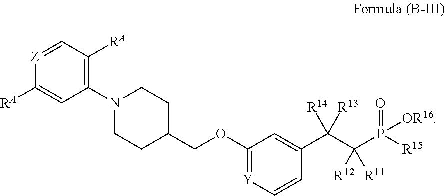

In some embodiments, the compound of Formula (B) is a compound of Formula (B-IV):

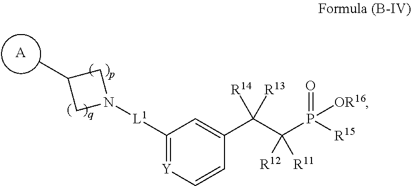

or a pharmaceutically acceptable salt, solvate, or stereoisomer thereof, wherein p and q are each independently 1 or 2.

In some embodiments, the compound of Formula (B-IV) is a compound of Formula (B-V) or Formula (B-VI), or a pharmaceutically acceptable salt, solvate, or stereoisomer thereof:

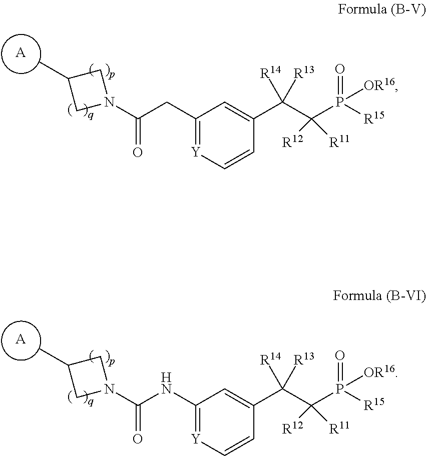

In some embodiments, Ring B is C₃-C₆ cycloalkylene; wherein the cycloalkylene is unsubstituted or substituted with 1, 2, 3, or 4 R^{B} substituents.

In some embodiments, the compound of Formula (B) is a compound of Formula (B-VII):

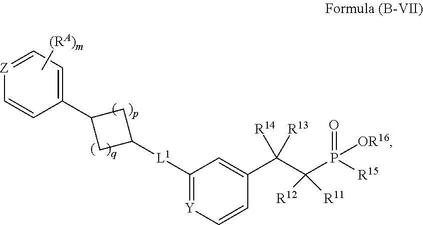

or a pharmaceutically acceptable salt, solvate, or stereoisomer thereof; wherein Z is N, CH, or CR^{A}; each R^{A} is independently —F, —Cl, C₁-C₇ alkyl, C₁-C₄ fluoroalkyl, —OH, or —OR¹⁰; p and q are each independently 1 or 2; and m is 0, 1, or 2.

In some embodiments, the compound of Formula (B-VII) is a compound of Formula (B-VIII), or a pharmaceutically acceptable salt, solvate, or stereoisomer thereof:

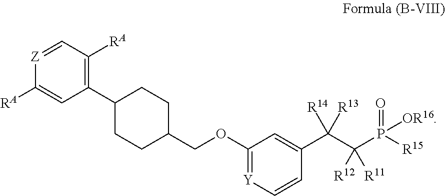

In some embodiments, R¹⁵ is methyl, ethyl, n-propyl, i-propyl, n-butyl, i-butyl, sec-butyl, or t-butyl. In some embodiments, R¹⁵ is methyl or ethyl. In some embodiments, R 15 is methyl. In some embodiments, R¹⁵ is ethyl.

In some embodiments, R¹⁶ is hydrogen, methyl, ethyl, n-propyl, i-propyl, n-butyl, i-butyl, sec-butyl, or t-butyl. In some embodiments, R¹⁶ is hydrogen, methyl, or ethyl. In some embodiments, R 16 is hydrogen.

In some embodiments, —P(═O)(R¹⁵) OR¹⁶ is —P(═O)(CH₃)OH or —P(═O)(CH₂CH₃)OH. In some embodiments, —P(═O)(R¹⁵) OR¹⁶ is —P(═O)(CH₃)OH. In some embodiments, —P(═O)(R¹⁵) OR¹⁶ is —P(═O)(CH₂CH₃)OH.

In some embodiments, the compound of Formula (B) that is useful for the methods described herein has a structure provided in Table 2.

TABLE 2

| Cpd. # | Structure | Name |
|---|---|---|
| B1* | 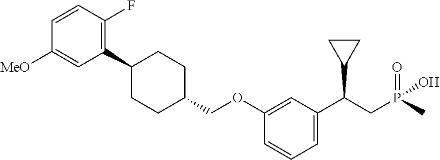 [SMILES: COc1ccc(F)c([C@H]2CC[C@H](COc3cccc([C@H](C[P@](C)(=O)O)C4CC4)c3)CC2)c1] | (R)-((R)-2-cyclopropyl-2-(3-(((1r,4R)-4-(2- fluoro-5- methoxyphenyl)cyclohexyl)methoxy)phenyl) ethyl)(methyl)phosphinic acid |
| B2* | 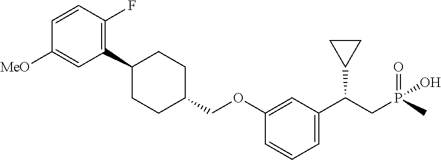 [SMILES: COc1ccc(F)c([C@H]2CC[C@H](COc3cccc([C@@H](C[P@](C)(=O)O)C4CC4)c3)CC2)c1] | (R)-((S)-2-cyclopropyl-2-(3-(((1r,4S)-4-(2- fluoro-5- methoxyphenyl)cyclohexyl)methoxy)phenyl) ethyl)(methyl)phosphinic acid |
| B3* | 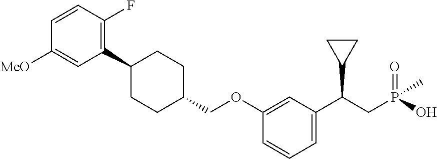 [SMILES: COc1ccc(F)c([C@H]2CC[C@H](COc3cccc([C@H](C[P@@](C)(=O)O)C4CC4)c3)CC2)c1] | (S)-((R)-2-cyclopropyl-2-(3-(((1r,4R)-4-(2- fluoro-5- methoxyphenyl)cyclohexyl)methoxy)phenyl) ethyl)(methyl)phosphinic acid |
| B4* | 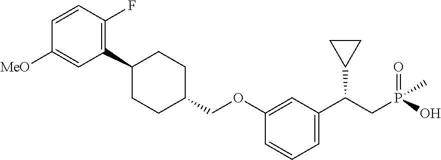 [SMILES: COc1ccc(F)c([C@H]2CC[C@H](COc3cccc([C@@H](C[P@@](C)(=O)O)C4CC4)c3)CC2)c1] | (S)-((S)-2-cyclopropyl-2-(3-(((1r,4S)-4-(2- fluoro-5- methoxyphenyl)cyclohexyl)methoxy)phenyl) ethyl)(methyl)phosphinic acid |
| B5 | 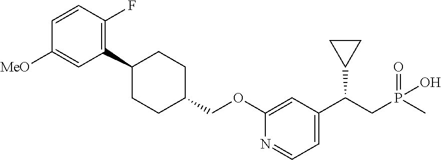 [SMILES: COc1ccc(F)c([C@H]2CC[C@H](COc3cc([C@@H](CP(C)(=O)O)C4CC4)ccn3)CC2)c1] | ((S)-2-cyclopropyl-2-(2-(((trans)-4-(2- fluoro-5- methoxyphenyl)cyclohexyl)methoxy)pyridin- 4-yl)ethyl)(methyl)phosphinic acid |
| B6 | 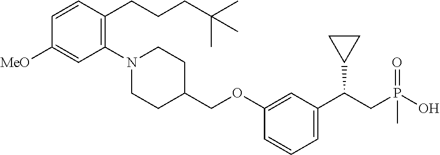 [SMILES: COc1ccc(CCCC(C)(C)C)c(N2CCC(COc3cccc([C@@H](CP(C)(=O)O)C4CC4)c3)CC2)c1] | ((S)-2-cyclopropyl-2-(3-((1-(2-(4,4- dimethylpentyl)-5-methoxyphenyl)piperidin- 4- yl)methoxy)phenyl)ethyl)(methyl)phosphinic acid |
| B7 | 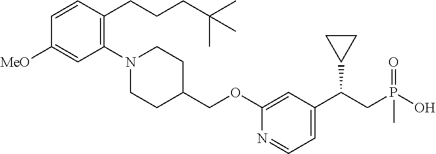 [SMILES: COc1ccc(CCCC(C)(C)C)c(N2CCC(COc3cc([C@@H](CP(C)(=O)O)C4CC4)ccn3)CC2)c1] | ((S)-2-cyclopropyl-2-(2-((1-(2-(4,4- dimethylpentyl)-5-methoxyphenyl)piperidin- 4-yl)methoxy)pyridin-4- yl)ethyl)(methyl)phosphinic acid |
| B8 | 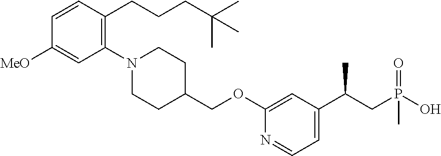 [SMILES: COc1ccc(CCCC(C)(C)C)c(N2CCC(COc3cc([C@@H](C)CP(C)(=O)O)ccn3)CC2)c1] | ((R)-2-(2-((1-(2-(4,4-dimethylpentyl)-5- methoxyphenyl)piperidin-4- yl)methoxy)pyridin-4- yl)propyl)(methyl)phosphinic acid |
| B9 | 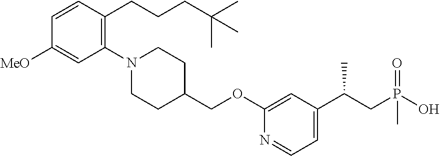 [SMILES: COc1ccc(CCCC(C)(C)C)c(N2CCC(COc3cc([C@H](C)CP(C)(=O)O)ccn3)CC2)c1] | ((S)-2-(2-((1-(2-(4,4-dimethylpentyl)-5- methoxyphenyl)piperidin-4- yl)methoxy)pyridin-4- yl)propyl)(methyl)phosphinic acid |
| B10 | 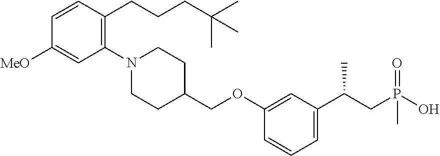 [SMILES: COc1ccc(CCCC(C)(C)C)c(N2CCC(COc3cccc([C@H](C)CP(C)(=O)O)c3)CC2)c1] | ((S)-2-(3-((1-(2-(4,4-dimethylpentyl)-5- methoxyphenyl)piperidin-4- yl)methoxy)phenyl)propyl)(methyl) phosphinic acid |
| B11 | 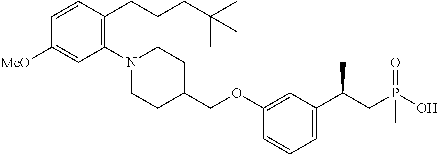 [SMILES: COc1ccc(CCCC(C)(C)C)c(N2CCC(COc3cccc([C@@H](C)CP(C)(=O)O)c3)CC2)c1] | ((R)-2-(3-((1-(2-(4,4-dimethylpentyl)-5- methoxyphenyl)piperidin-4- yl)methoxy)phenyl)propyl)(methyl) phosphinic acid |
| B12 | 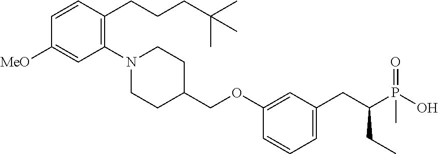 [SMILES: CC[C@@H](Cc1cccc(OCC2CCN(c3cc(OC)ccc3CCCC(C)(C)C)CC2)c1)P(C)(=O)O] | ((S)-1-(3-((1-(2-(4,4-dimethylpentyl)-5- methoxyphenyl)piperidin-4- yl)methoxy)phenyl)butan-2- yl)(methyl)phosphinic acid |
| B13 | 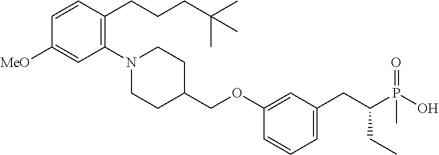 [SMILES: CC[C@H](Cc1cccc(OCC2CCN(c3cc(OC)ccc3CCCC(C)(C)C)CC2)c1)P(C)(=O)O] | ((R)-1-(3-((1-(2-(4,4-dimethylpentyl)-5- methoxyphenyl)piperidin-4- yl)methoxy)phenyl)butan-2- yl)(methyl)phosphinic acid |

* absolute stereochemistry unknown, stereoisomers separated by chiral purification

In some embodiments, the compound of Formula (B) that is useful for the methods described herein is a pharmaceutically acceptable salt of a compound in Table 2.

### Further Forms of Compounds

Furthermore, in some embodiments, the compounds described herein exist as “geometric isomers.” In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

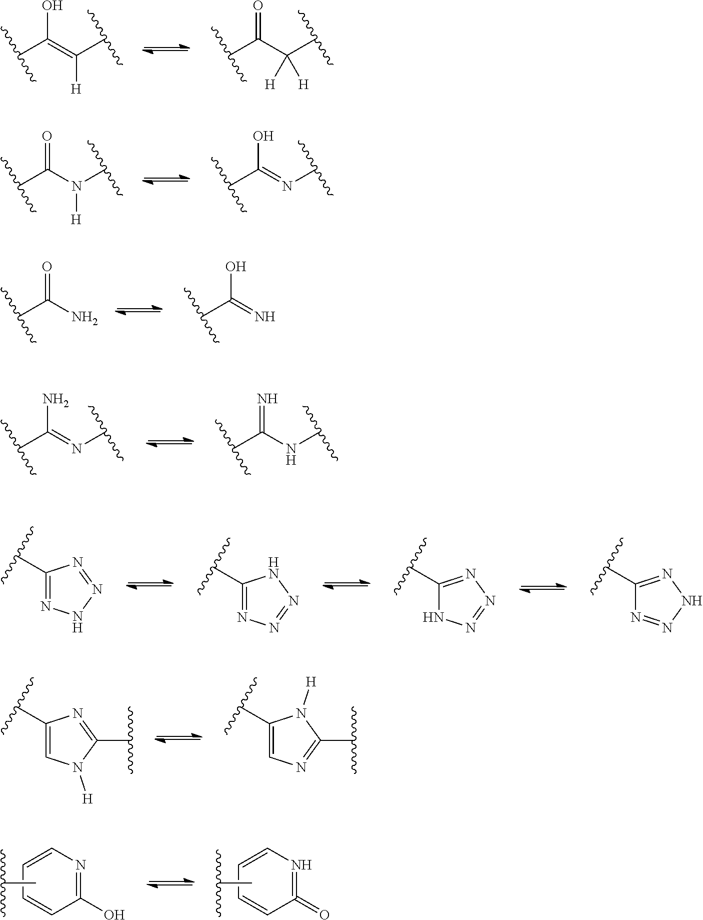

In some situations, the compounds described herein possess one or more chiral centers and each center exists in the (R)-configuration or(S)-configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

The term “positional isomer” refers to structural isomers around a central ring, such as ortho-, meta-, and para-isomers around a benzene ring.

The methods and formulations described herein include the use of N-oxides (if appropriate), crystalline forms (also known as polymorphs), or pharmaceutically acceptable salts of compounds described herein, as well as active metabolites of these compounds having the same type of activity.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. “Hydrates” are formed when the solvent is water, or “alcoholates” are formed when the solvent is alcohol. Solvates of compounds described herein are conveniently prepared or formed during the processes described herein. The compounds provided herein optionally exist in either unsolvated as well as solvated forms.

The compounds disclosed herein, in some embodiments, are used in different enriched isotopic forms, e.g., enriched in the content of ²H, ³H, ¹¹C, ¹³C and/or ¹⁴C. In some embodiments, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ³H, ¹¹C, ¹³C, ¹⁴C, ¹⁵C, ¹²N, ¹³N, ¹⁵N, ¹⁶N, ¹⁷O, ¹⁸O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the 1H atoms replaced with 2H atoms. The methods of synthesis for deuterium-containing compounds are known in the art. In some embodiments deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

In some embodiments, the compounds described herein are labeled by other means, including, but not limited to, the use of chromophores or fluorescent moieties, bioluminescent labels, or chemiluminescent labels.

In certain embodiments, the compounds described herein, or a pharmaceutically acceptable salt, solvate, stereoisomer, or prodrug thereof, as described herein are substantially pure, in that it contains less than about 5%, or less than about 1%, or less than about 0.1%, of other organic small molecules, such as contaminating intermediates or by-products that are created, for example, in one or more of the steps of a synthesis method.

### Preparation of the Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions.

The preparation of Compounds of Formula (A) has been described in WO 2021/071837 and US 2022/0153719, each of which is incorporated by reference for such disclosure. The preparation of Compounds of Formula (B) has been described in WO 2021/174048 and U.S. application Ser. No. 17/745,126, each of which is incorporated by reference for such disclosure.

### Definitions

The term “modulate” or “modulating” or “modulation” refers to an increase or decrease in the amount, quality, or effect of a particular activity, function or molecule. By way of illustration and not limitation, agonists, inverse agonists, antagonists, and allosteric modulators of a G protein-coupled receptor are modulators of the receptor.

The term “agonism” as used herein refers to the activation of a receptor or enzyme by a modulator, or agonist, to produce a biological response.

The term “agonist” as used herein refers to a modulator that binds to a receptor or enzyme and activates the receptor to produce a biological response. By way of example only, “GPR119 agonist” can be used to refer to a compound that exhibits an EC₅₀ with respect to GPR119activity of no more than about 100 μM, as measured in the cAMP production assay and glucagon-like peptide-1 (GLP-1) secretion assays. In some embodiments, the term “agonist” includes full agonists or partial agonists.

The term “full agonist” refers to a modulator that binds to and activates a receptor with the maximum response that an agonist can elicit at the receptor.

The term “partial agonist” refers to a modulator that binds to and activates a given receptor, but has partial efficacy, that is, less than the maximal response, at the receptor relative to a full agonist.

The term “positive allosteric modulator” refers to a modulator that binds to a site distinct from the orthosteric binding site and enhances or amplifies the effect of an agonist.

The term “antagonism” as used herein refers to the inactivation of a receptor or enzyme by a modulator, or antagonist. Antagonism of a receptor, for example, is when a molecule binds to the receptor and does not allow activity to occur.

The term “antagonist” or “neutral antagonist” as used herein refers to a modulator that binds to a receptor or enzyme and blocks a biological response. An antagonist has no activity in the absence of an agonist or inverse agonist but can block the activity of either, causing no change in the biological response.

The term “inverse agonist” refers to a modulator that binds to the same receptor as an agonist but induces a pharmacological response opposite to that agonist, i.e., a decrease in biological response.

The term “negative allosteric modulator” refers to a modulator that binds to a site distinct from the orthosteric binding site and reduces or dampens the effect of an agonist.

As used herein, “EC₅₀” is intended to refer to the concentration of a substance (e.g., a compound or a drug) that is required for 50% activation or enhancement of a biological process. In some instances, EC₅₀ refers to the concentration of agonist that provokes a response halfway between the baseline and maximum response in an in vitro assay. In some embodiments as used herein, EC₅₀ refers to the concentration of a modulator (e.g., an agonist) that is required for 50% activation of a GPCR, for example, GPR40 or GPR119.

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an antagonist or inhibitor) that is required for 50% inhibition of a receptor, for example, SSTR5, or an enzyme, for example, DPP-4, or PDE4.

The terms “subject,” “individual,” and “patient” are used interchangeably. These terms encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “gut-restricted” as used herein refers to a compound, e.g., a receptor modulator, that is predominantly active in the gastrointestinal system. In some embodiments, the biological activity of the gut-restricted compound, e.g., a gut-restricted receptor modulator, is restricted to the gastrointestinal system. In some embodiments, gastrointestinal concentration of a gut-restricted modulator, is higher than the IC₅₀ value or the EC₅₀ value of the gut-restricted modulator against its receptor, while the plasma levels of said gut-restricted modulator are lower than the IC₅₀ value or the EC₅₀ value of the gut-restricted modulator against its receptor. In some embodiments, the gut-restricted compound, e.g., a receptor modulator, is non-systemic. In some embodiments, the gut-restricted compound, e.g., a receptor modulator, is a non-absorbed compound. In other embodiments, the gut-restricted compound is minimally absorbed and rapidly metabolized to metabolites that are significantly less active than the modulator itself toward the target receptor.

In some embodiments, the gut-restricted modulator is non-systemic but is instead localized to the gastrointestinal system. In some instances, the modulator is present in high levels in the gut, but low levels in serum. In some embodiments, the systemic exposure of a gut-restricted modulator is, for example, less than 100, less than 50, less than 20, less than 10, or less than 5 nM, bound or unbound, in blood serum. In some embodiments, the intestinal exposure of a gut-restricted modulator is, for example, greater than 1000, 5000, 10000, 50000, 100000, or 500000 nM. In some embodiments, a modulator is gut-restricted due to poor absorption of the modulator itself, or because of absorption of the modulator which is rapidly metabolized in serum resulting in low systemic circulation, or due to both poor absorption and rapid metabolism in the serum. In some embodiments, a modulator is covalently bonded to a kinetophore, optionally through a linker, which changes the pharmacokinetic profile of the modulator. In other embodiments, two or more modulators are covalently bonded, optionally through a linker, to each other.

The term “kinetophore” as used herein refers to a structural unit tethered to a small molecule modulator, optionally through a linker, which makes the whole molecule larger and increases the polar surface area while maintaining biological activity of the small molecule modulator. The kinetophore influences the pharmacokinetic properties, for example solubility, absorption, distribution, rate of elimination, and the like, of the small molecule modulator and has minimal changes to the binding to or association with a receptor. The defining feature of a kinetophore is not its interaction with the target, for example a receptor, but rather its effect on specific physiochemical characteristics of the modulator to which it is attached. In some instances, kinetophores are used to restrict a modulator to the gut.

The term “linked” as used herein refers to a covalent linkage between a modulator and a kinetophore or between a modulator and at least one other modulator, or a combination thereof. The linkage can be through a covalent bond, or through a “linker.” As used herein, “linker” refers to one or more bifunctional molecules which can be used to covalently bond to the modulator(s) and/or kinetophore. In some embodiments, the linker is attached to any part of the modulator so long as the point of attachment does not interfere with the binding of the modulator to its receptor. In some embodiments, the linker is non-cleavable. In some embodiments, the linker is cleavable. In some embodiments, the linker is cleavable in the gut. In some embodiments, cleaving the linker releases the biologically active modulator in the gut.

The term “gastrointestinal system” (GI system) or “gastrointestinal tract” (GI tract) as used herein, refers to the organs and systems involved in the process of digestion. The gastrointestinal tract includes the esophagus, stomach, small intestine, which includes the duodenum, jejunum, and ileum, and large intestine, which includes the cecum, colon, and rectum. In some embodiments herein, the GI system refers to the “gut,” meaning the stomach, small intestines, and large intestines or to the small and large intestines, including, for example, the duodenum, jejunum, and/or colon.

### Pharmaceutical Compositions

In some embodiments, disclosed herein is a pharmaceutical composition comprising a compound of Formula (A), or a pharmaceutically acceptable salt, solvate, or stereoisomer thereof, and the compound of Formula (B), or a pharmaceutically acceptable salt, solvate, or stereoisomer thereof, and a pharmaceutically acceptable excipient.

In some embodiments, the modulators are combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

### EXAMPLES

The following examples are provided for illustrative purposes only and not to limit the scope of the claims provided herein.

### Example 1. In Vitro Activity Assays

### Example 1A: GPR119

### Cell Line Expressing GPR119

CHO-K1 cells stably expressing human GPR119 (hGPR119) were prepared by transfection of a GPR119-carrying plasmid using Lipofectamine 2000 (following manufacturer instructions). A stable cell line was established using the limiting dilution method with geneticine selection. Assay-ready frozen (ARF) cells were prepared and used throughout the study.

### CAMP Accumulation Assay

The assay was performed in a 384-well plate format using the CAMP Gs dynamic assay kit from Cisbio. ARF cells expressing hGPR119 were thawed, washed and then resuspended in CAMP stimulation buffer at a cell density of 1.1×10⁶ cells/mL. Cells were plated at a density of ~10,000 cells/well (9 μL/well). Dose response curves for the tested compounds were prepared in a cAMP stimulation buffer, containing 0.1% Tween 80 at 4 fold the final concentration. The compounds were then transferred to the cell plates using BRAVO (3 μL/well) and the plates were incubated for 60 minutes at 37° C./5% CO₂. Detection buffer (10 μL, prepared as described in the CAMP Gs dynamic kit) were added to each well, and the plates were incubated at ambient temperature for 1 hr.

RT-FRET was measured using a ClarioSTAR plate reader, calculating the ratio between emissions at 665 nm and 620 nm (HTRF ratio). The HTRF ratio for positive (Max) and negative (Min) controls were used to normalize HTRF data and generate values for % activity. EC₅₀ and Max activity values were determined using a standard 4-parameter fit.

Results for exemplary compounds are shown in the following Table.

| Compound | EC₅₀^{a} |
|---|---|
| A1 | A |
| A2 | B |
| A3 | B |
| A4 | A |
| A5 | A |
| A6 | B |
| A7 | B |
| A8 | B |
| A9 | A |
| A10 | B |
| A11 | B |
| A12 | B |
| A13 | B |
| A14 | B |
| A15 | A |
| A16 | A |
| A17 | A |
| A18 | A |
| A19 | A |
| A20 | A |
| A21 | B |
| A22 | A |
| A23 | A |
| A24 | B |
| A25 | A |
| A26 | A |
| A27 | A |
| A28 | B |
| A29 | B |
| A30 | B |
| A31 | B |
| A32 | B |
| A33 | A |
| A34 | A |
| A35 | A |
| A36 | A |
| A37 | A |
| A38 | A |
| A40 | A |
| A41 | A |
| A42 | A |
| A43 | B |
| A44 | B |
| A45 | A |
| A46 | A |
| A47 | B |
| A48 | A |
| A49 | B |
| A50 | B |
| A51 | B |
| A52 | B |
| A53 | B |
| A54 | B |
| A55 | C |
| A56 | B |
| A57 | A |
| A58 | A |
| A59 | A |
| A60 | A |
| A61 | A |
| A62 | A |
| A63 | B |
| A64 | A |
| A65 | B |
| A66 | B |
| A67 | B |
| A68 | B |
| A69 | B |
| A70 | B |
| A71 | A |
| A72 | A |

^{a}A ≤ 100 nM; 100 nM < B ≤ 1000 nM; 1000 nM < C ≤ 10000 nM.

### Example 1B: GPR40

### Cell Lines Expressing GPR40/FFAR1

CHO-K1 cells expressing human GPR40 were purchased from DiscoverX (95-1005C₂). HEK293 cells expressing mouse FFAR1 were prepared using a mouse FFAR1 carrying plasmid purchased from OriGene Technologies (MR222997). The cells were transfected using Lipofectamine 2000 using manufacturer instructions and stable cell line was established from a single cell using geneticine selection. Assay ready frozen (ARF) cells were prepared and used throughout the study.

### Inositol Phosphate Accumulation Assay

The assay was performed in a 384-well plate format using IP1 assay kit from Cis-Bio. ARF cells expressing FFAR1 (mouse and human) were thawed, washed and then plated in the appropriate medium (F12 based medium for CHO hFFAR1 and DMEM based medium for HEK293 mFFAR1—both were supplemented with 10% FBS and penicillin/streptomycin). 20 μL of 3.5×10⁵ cells/mL were plated on a Poly D-Lysine coated 384-well white plate. The cells were then incubated for 16 hr at 37° C./5% CO₂. After 16 hr the medium was removed and 15 μL of stimulation buffer containing the test compounds was added to the cells. The plates were then incubated for 90 min at 37° C./5% CO₂. 5 μL of detection buffer (prepared as described in the IP-one kit) was added to each well and the plates were incubated at RT for 1 hr.

RT-FRET was measured using ClarioSTAR plate reader, calculating the ratio between emissions at 665 nm and 620 nm (HTRF ratio). HTRF ratio for positive (Max) and negative (Min) controls were used to normalize HTRF data and generate values for % activity. EC₅₀ and Max activity values were determined using a standard 4-parameter fit.

Results for exemplary compounds are shown in the following table.

| Compound | Human EC₅₀^{a} |
|---|---|
| B1 | A |
| B2 | B |
| B3 | A |
| B4 | B |
| B5 | A |
| B6 | A |
| B7 | A |
| B8 | A |
| B9 | C |
| B10 | A |
| B11 | B |
| B12 | C |
| B13 | C |

^{a}A ≤ nM; 50 nM < B ≤ 250 nM; 250 nM < C ≤ 1000 nM.

### Example 2. Effect of Compounds Alone and in Combination on GLP-1 Secretion in Human Ileal Crypt Cultures

The potential for the combination of a compound of Formula (A) and a compound of Formula (B) to induce gut hormone secretion from EECs was assessed using primary human ileal crypt cultures and compared to the effect of the individual agents.

A compound of Formula (A) and a compound of Formula (B) each independently induced a roughly two-fold increase in GLP-1. The combination resulted in greater secretion of GLP-1.

Results for exemplary compounds are shown in the following table.

| Compound(s) | GLP-1 |
|---|---|
| (30 μM) | (fold increase) |
| A14 | 1.9 |
| B1 | 5.5 |
| A14 + B1 | 14.6 |
| B6 | 4.0 |
| A14 + B6 | 13.9 |

### Example 3. Gut Hormone Secretion in Mice with Compounds Alone and in Combination

To determine whether activating two receptors simultaneously results in increased effects on hormone release, representative GPR40 agonists of Formula (B) were tested in combination with a GPR119 agonist of Formula (A). GLP-1 was measured at 5 and 17 h post dose. For the dose titration study, male C57BL/6J mice were acclimated to the facility and the dosing procedure. Mice were weighed and then fasted for 4 h. The mice were administered vehicle, a GPR40 agonist of Formula (B) at 30 mg/kg, a GPR119 agonist of Formula (A) at 30 mg/kg, and the GPR40 agonist of Formula (B) at 0.3, 1, 3, 10, and 30 mg/kg doses in combination with the GPR119 agonist of Formula (A) at 30 mg/kg by oral gavage. Blood was collected at 5 h post-dose or 17 h post-dose, and plasma was assayed for GLP-1.

Each compound dosed alone at 30 mg/kg led to modest but significant release of GLP-1 when compared to vehicle at 5 hr post dose. However, when the GPR40 agonist of Formula (B) at a maximal dose was administered with a fixed maximal dose of the GPR119 agonist of Formula (A), larger increases in GLP-1 secretion were observed.

Similar additive increases in GLP-1 secretion were observed at a later timepoint. At 17 h after compound administration, higher doses were required to achieve maximal efficacy.

Results for exemplary compounds are shown in the following table. The data displayed corresponds to administration of doses of 30 mg/kg of each of the compounds listed.

| Timepoint | | GLP-1 |
|---|---|---|
| (h) | Compound(s) | (fold increase) |
| 5 | A14 | 1.6 |
| 5 | B1 | 1.9 |
| 5 | A14 + B1 | 4.8 |
| 5 | B6 | 3.2 |
| 5 | A14 + B6 | 6.2 |
| 17 | A14 | 1.3 |
| 17 | B1 | 1.4 |
| 17 | A14 + B1 | 5.1 |
| 17 | B6 | 1.9 |
| 17 | A14 + B6 | 4.6 |

### Example 4. Effect of a GPR40 Agonist of Formula (B) Alone or in Combination with a GPR119 Agonist of Formula (A) on 14-Day Weight Loss in DIO Mice

The effects of a GPR40 Agonist of Formula (B) alone and in combination with a GPR119 Agonist of Formula (A) on body weight were assessed in diet-induced-obese (DIO) mice over 14 days.

Male C57BL/6J DIO mice, approximately 16 weeks old (Jackson Laboratories), were provided 60% high fat diet and water ad libitum. Animals were dosed BID (two times a day) with vehicle for an approximately 3-week until body weight had stabilized. At approximately 20 weeks of age, the mice were randomized into treatment groups (n=8/group) by body weight (range of 37 to 55 g) as well as food intake, and body weight change measured over a two-day baseline period. In order to maximize time on target throughout the day, compounds were administered BID, 8 h apart. Food intake and body weight were measured daily.

Across two parallel studies, the combination of a compound of Formula (A) and a compound of Formula (B) resulted in >10% body weight loss vs. vehicle. In these parallel studies, the compounds were administered for 13 or 14 days. The GPR119 agonist of Formula (A) as a single agent does not cause appreciable weight loss and the GPR40 agonist of Formula (B) alone results in less than approximately half the weight loss observed with the combination. Results for exemplary compounds and combinations are shown in the following table.

| | | % Weight Loss vs |
|---|---|---|
| Study # | Compound(s) | Vehicle |
| 1 | A14 | 2.5 |
| 1 | B1 | 3.1 |
| 1 | A14 + B1 | 11.3 |
| 2 | B6 | 5.2 |
| 2 | A14 + B6 | 14.1 |

### Example 5. Effects of a GPR40 Agonist of Formula (B) Alone and in Combination with Other Agents on Glucose Excursion in Mice

The effects of GPR40 agonists of Formula (B) and a GPR119 agonist of Formula (A) alone and in combination on glucose excursion during an oral glucose tolerance test were assessed in mice. The effects of the combination of a compound of Formula (A) and compounds of Formula (B) with linagliptin (a DPP-4 inhibitor) or metformin were also assessed.

Male C57BL/6J mice, approximately 10 weeks old, were acclimated to the facility and the oral dosing procedure. Mice were randomized into balanced treatment groups using body weight. Baseline glucose was assessed immediately before compound administration. One hour after compound dosing, glucose levels were again assessed immediately prior to oral administration of D-(+)-glucose (5 g/kg in water) at 10 mL/kg. Blood was assessed for glucose levels 20 min, 40 min, 60 min, and 120 min post D-(+)-glucose administration. The AUCs for blood glucose measures were calculated for each treatment from 0 to 120 min. Statistical analysis was performed on AUCs using one-way ANOVA versus vehicle, with Tukey's post-test.

A compound of Formula (A), compounds of Formula (B), linagliptin, and metformin alone all suppressed post-prandial glucose excursion. The combination of a compound of Formula (A) and compounds of Formula (B) resulted in a greater reduction in post-prandial glucose excursion compared to the individual agents (Table 4). Similarly, the combination of a compound of Formula (A) and compounds of Formula (B) with linagliptin or metformin resulted in greater glucose control compared to the combination of a compound of Formula (A) and compounds of Formula (B) alone.

Results for exemplary compounds and combinations are shown in the following table.

| | | | | | % |
|---|---|---|---|---|---|
| | | | | | Reduction |
| | | | | | in 2-h |
| | | | | | glucose |
| B6 | B1 | A14 | linagliptin | metformin | AUC vs. |
| (mg/kg) | (mg/kg) | (mg/kg) | (mg/kg) | (mg/kg) | vehicle |
| 0 | 0 | 0 | 0.3 | 0 | 17 |
| 0 | 0 | 0 | 0 | 300 | 26 |
| 0 | 0 | 30 | 0 | 0 | 14 |
| 30 | 0 | 0 | 0 | 0 | 21 |
| 30 | 0 | 30 | 0 | 0 | 33 |
| 30 | 0 | 30 | 0.3 | 0 | 38 |
| 0 | 30 | 0 | 0 | 0 | 14 |
| 0 | 30 | 30 | 0 | 0 | 34 |
| 0 | 30 | 30 | 0.3 | 0 | 36 |
| 0 | 30 | 30 | 0 | 300 | 50 |

The examples and embodiments described herein are for illustrative purposes only and various modifications or changes suggested to persons skilled in the art are to be included within the spirit and purview of this application and scope of the appended claims.

What is claimed is:

1. A method of treating a condition or disorder involving the gut-brain axis in an individual in need thereof, the method comprising administering to the individual:
i) a compound of Formula (A): 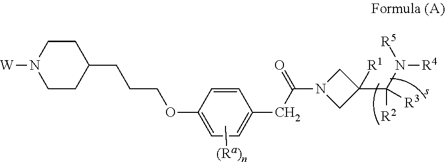
or a pharmaceutically acceptable salt, solvate, or stereoisomer thereof, wherein:
R¹ is hydrogen, —OH, or C₁₋₈ alkyl, wherein the alkyl is unsubstituted or substituted by —OH or —O(C₁₋₆ alkyl);
each R² and R³ is hydrogen;
or R² and R³ on the same carbon atom are taken together to form ═O;
R⁴ is hydrogen or C₁₋₆ alkyl;
R⁵ is C₁₋₈ alkyl, C₃₋₈ cycloalkyl, 4- to 8-membered heterocycloalkyl, —[(CH₂)ᵣ—Z]ₜ—R⁶, —[(CHR^{d})ᵣ—Z]ₜ—R⁶, or —[(C(R^{d})₂)ᵣ—Z]ₜ—R⁶; wherein each alkyl, cycloalkyl, and 4- to 8-membered heterocycloalkyl is substituted by 1-6 R^{c} groups each Z is independently —CH₂O—, —CH₂NR⁴, —CH₂N⁺(R^{d})₂—, or —NH—C(═O)—NH—;
each r is independently 1-6;
each t is independently 1-6;
R⁶ is hydrogen, C₁₋₈ alkyl, C₃₋₈ cycloalkyl, or 4- to 8-membered heterocycloalkyl, wherein the alkyl, cycloalkyl, or 4- to 8-membered heterocycloalkyl is unsubstituted or substituted by 1-6 R^{c} groups;
or R⁴ and R⁵ are taken together with the nitrogen to which they are attached to form a 4- to 8-membered heterocycloalkyl, which is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH) 2, —P(═O)(OH)(R^{d}), —P(═O)(OH)(OR^{d}), 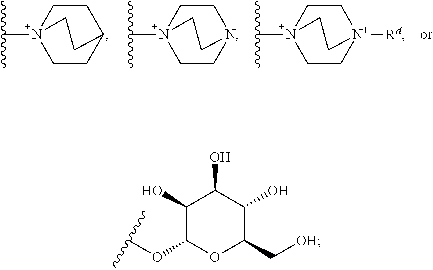
each R^{d} is independently C₁₋₆ alkyl;
each R^{a} is independently halogen, —CN, C₁₋₆ alkyl, C₁₋₆ fluoroalkyl, or C₃-6 cycloalkyl;
W is phenyl or 5-6 membered monocyclic heteroaryl, wherein the phenyl or heteroaryl is unsubstituted or substituted with 1, 2, or 3 substituents selected from R^{b};
each R^{b} is independently halogen, —OH, —CN, —C(O)OH, —C(O)O(C₁₋₆ alkyl), C₁₋₆ alkyl, C₁₋₆ alkoxy, C₃-6 cycloalkyl, phenyl, or 5- to 6-membered heteroaryl; wherein each alkyl, alkoxy, and cycloalkyl is unsubstituted or substituted with 1, 2, or 3 substituents selected from halogen, —OH, C₁₋₆ alkyl, and C₁₋₆ alkoxy;
n is 0-4; and
s is 1 or 2;
and
ii) a compound of Formula (B): 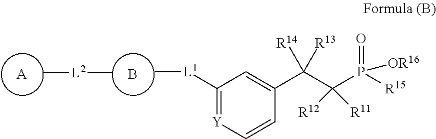
or a pharmaceutically acceptable salt, solvate, or stereoisomer thereof, wherein:
R¹¹, R¹², and R¹³ are each independently hydrogen, halogen, or C₁-C₆ alkyl;
R¹⁴ is hydrogen, C₁-C₆ alkyl, or C₃-C₆ cycloalkyl;
R¹⁵ is C₁-C₆ alkyl;
R¹⁶ is hydrogen or C₁-C₆ alkyl;
Y is CH or N;
L¹ is *—O—CH₂—, *—CH₂—O—, *—NR¹⁷—CH₂—, *—NR¹⁷—C(O)—, *—C(O)—NR¹⁷—, or *—C(O)—CH₂—;
wherein * represents the connection to Ring B;
R¹⁷ is hydrogen or C₁-C₆ alkyl;
Ring B is 3- to 6-membered heterocycloalkylene; wherein the heterocycloalkylene is unsubstituted or substituted with 1, 2, 3, or 4 R^{B} substituents;
or Ring B is C₃-C₆ cycloalkylene; wherein the cycloalkylene is unsubstituted or substituted with 1, 2, 3, or 4 R^{B} substituents;
each R^{B} is independently halogen, C₁-C₆ alkyl, or C₁-C₆ fluoroalkyl;
L² is a bond or C₁-C₆ alkylene; wherein the alkylene is unsubstituted or substituted with 1, 2, or 3 substituents selected from the group consisting of —OH, C₁-C₆ alkyl, and —O—(C₁-C₆ alkyl);
Ring A is aryl or heteroaryl; wherein the aryl or heteroaryl is unsubstituted or substituted with 1, 2, or 3 R^{A} substituents;
each R^{A} is independently —F, —Cl, C₁-C₁₀ alkyl, C₁-C₁₀ fluoroalkyl, C₁-C₁₀ hydroxyalkyl, —OH, —OR¹⁸, —(C₁-C₆ alkylene)—OR¹⁸, —N(R¹⁹)₂, —(C₁-C₆ alkylene)-N(R¹⁹)₂;
each R¹⁸ is independently C₁-C₆ alkyl; wherein each alkyl is independently unsubstituted or substituted with 1, 2, 3, 4, or 5 substituents selected from the group consisting of halogen, —OH, C₁-C₆ alkyl and C₁-C₆ hydroxyalkyl; and
each R¹⁹ is independently hydrogen, C₁-C₆ alkyl, or monocyclic heteroaryl; wherein each alkyl and heteroaryl is independently unsubstituted or substituted with 1, 2, 3, 4, or 5 substituents selected from the group consisting of halogen, —OH, C₁-C₆ alkyl and C₁-C₆ hydroxyalkyl;
or two R¹⁹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

2. The method of claim 1, wherein the condition involving the gut-brain axis is a metabolic disorder.

3. The method of claim 2, wherein the metabolic disorder is type 2 diabetes, hyperglycemia, metabolic syndrome, obesity, hypercholesterolemia, or nonalcoholic steatohepatitis.

4. The method of claim 2, wherein the metabolic disorder is type 2 diabetes.

5. The method of claim 2, wherein the metabolic disorder is obesity.

6. The method of claim 1, wherein the condition involving the gut-brain axis is a nutritional disorder.

7. The method of claim 6, wherein the nutritional disorder is short bowel syndrome, intestinal failure, or intestinal insufficiency.

8. The method of claim 6, wherein the nutritional disorder is short bowel syndrome.

9. The method of claim 1, wherein the condition involving the gut-brain axis is an eating disorder.

10. The method of claim 9, wherein the eating disorder is hyperphagia, anorexia nervosa, or binge eating disorder.

11. The method of claim 9, wherein the eating disorder is binge eating disorder.

12. A method of weight management in an individual in need thereof comprising administering to the individual:
i) a compound of Formula (A): 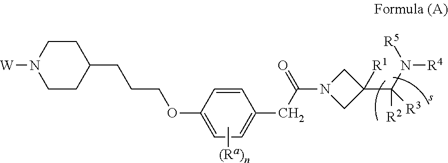
or a pharmaceutically acceptable salt, solvate, or stereoisomer thereof, wherein:
R¹ is hydrogen, —OH, or C₁₋₈ alkyl, wherein the alkyl is unsubstituted or substituted by —OH or —O(C₁₋₆ alkyl);
each R² and R³ is hydrogen;
or R² and R³ on the same carbon atom are taken together to form ═O;
R⁴ is hydrogen or C₁₋₆ alkyl;
R⁵ is C₁₋₈ alkyl, C₃₋₈ cycloalkyl, 4- to 8-membered heterocycloalkyl, —[(CH₂)ᵣ—Z]ₜ—R⁶, —[(CHR^{d})ᵣ—Z]ₜ—R⁶, or —[(C(R^{d})₂)ᵣ—Z]ₜ—R⁶; wherein each alkyl, cycloalkyl, and 4- to 8-membered heterocycloalkyl is substituted by 1-6 R^{c} groups
each Z is independently —CH₂O—, —CH₂NR^{d}, —CH₂N⁺(R^{d})₂—, or —NH—C(═O)—NH—;
each r is independently 1-6;
each t is independently 1-6;
R⁶ is hydrogen, C₁₋₈ alkyl, C₃₋₈ cycloalkyl, or 4- to 8-membered heterocycloalkyl, wherein the alkyl, cycloalkyl, or 4- to 8-membered heterocycloalkyl is unsubstituted or substituted by 1-6 R^{c} groups;
or R⁴ and R⁵ are taken together with the nitrogen to which they are attached to form a 4- to 8-membered heterocycloalkyl, which is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH) 2, —P(═O)(OH)(R^{d}), —P(═O)(OH)(OR^{d}), 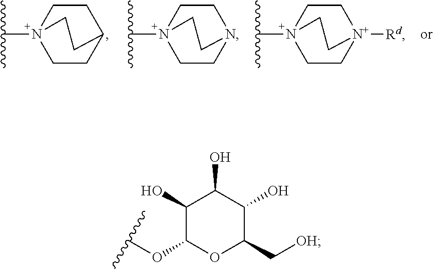
each R^{d} is independently C₁₋₆ alkyl;
each R^{a} is independently halogen, —CN, C₁₋₆ alkyl, C₁₋₆ fluoroalkyl, or C₃-6 cycloalkyl;
W is phenyl or 5-6 membered monocyclic heteroaryl, wherein the phenyl or heteroaryl is unsubstituted or substituted with 1, 2, or 3 substituents selected from R^{b};
each R^{b} is independently halogen, —OH, —CN, —C(O)OH, —C(O)O(C₁₋₆ alkyl), C₁₋₆ alkyl, C₁₋₆ alkoxy, C₃-6 cycloalkyl, phenyl, or 5- to 6-membered heteroaryl; wherein each alkyl, alkoxy, and cycloalkyl is unsubstituted or substituted with 1, 2, or 3 substituents selected from halogen, —OH, C₁₋₆ alkyl, and C₁₋₆ alkoxy;
n is 0-4; and
s is 1 or 2;
and
ii) a compound of Formula (B): 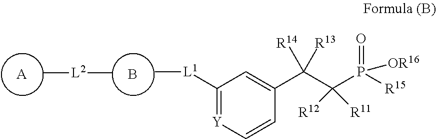
or a pharmaceutically acceptable salt, solvate, or stereoisomer thereof, wherein:
R¹¹, R¹², and R¹³ are each independently hydrogen, halogen, or C₁-C₆ alkyl;
R¹⁴ is hydrogen, C₁-C₆ alkyl, or C₃-C₆ cycloalkyl;
R¹⁵ is C₁-C₆ alkyl;
R¹⁶ is hydrogen or C₁-C₆ alkyl;
Y is CH or N;
L¹ is *—O—CH₂—, *—CH₂—O—, *—NR¹⁷—CH₂—, *—NR¹⁷—C(O)—, *—C(O)—NR¹⁷—, or *—C(O)—CH₂—;
wherein * represents the connection to Ring B;
R¹⁷ is hydrogen or C₁-C₆ alkyl;
Ring B is 3- to 6-membered heterocycloalkylene; wherein the heterocycloalkylene is unsubstituted or substituted with 1, 2, 3, or 4 R^{B} substituents;
or Ring B is C₃-C₆ cycloalkylene; wherein the cycloalkylene is unsubstituted or substituted with 1, 2, 3, or 4 R^{B} substituents;
each R^{B} is independently halogen, C₁-C₆ alkyl, or C₁-C₆ fluoroalkyl;
L² is a bond or C₁-C₆ alkylene; wherein the alkylene is unsubstituted or substituted with 1, 2, or 3 substituents selected from the group consisting of —OH, C₁-C₆ alkyl, and —O—(C₁-C₆ alkyl);
Ring A is aryl or heteroaryl; wherein the aryl or heteroaryl is unsubstituted or substituted with 1, 2, or 3 R^{A} substituents;
each R^{A} is independently —F, —Cl, C₁-C₁₀ alkyl, C₁-C₁₀ fluoroalkyl, C₁-C₁₀ hydroxyalkyl, —OH, —OR¹⁸, —(C₁-C₆ alkylene)—OR¹⁸, —N(R¹⁹)₂, —(C₁-C₆ alkylene)-N(R¹⁹)₂;
each R¹⁸ is independently C₁-C₆ alkyl; wherein each alkyl is independently unsubstituted or substituted with 1, 2, 3, 4, or 5 substituents selected from the group consisting of halogen, —OH, C₁-C₆ alkyl and C₁-C₆ hydroxyalkyl; and
each R¹⁹ is independently hydrogen, C₁-C₆ alkyl, or monocyclic heteroaryl; wherein each alkyl and heteroaryl is independently unsubstituted or substituted with 1, 2, 3, 4, or 5 substituents selected from the group consisting of halogen, —OH, C₁-C₆ alkyl and C₁-C₆ hydroxyalkyl;
or two R¹⁹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

13. The method of claim 12, wherein said weight management comprises one or more of: weight loss, maintenance of weight loss, decreased food consumption, increasing meal-related satiety, reducing pre-meal hunger, and reducing intra-meal food intake.

14. The method of any one of claims 1-13, wherein the compound of Formula (A) is: 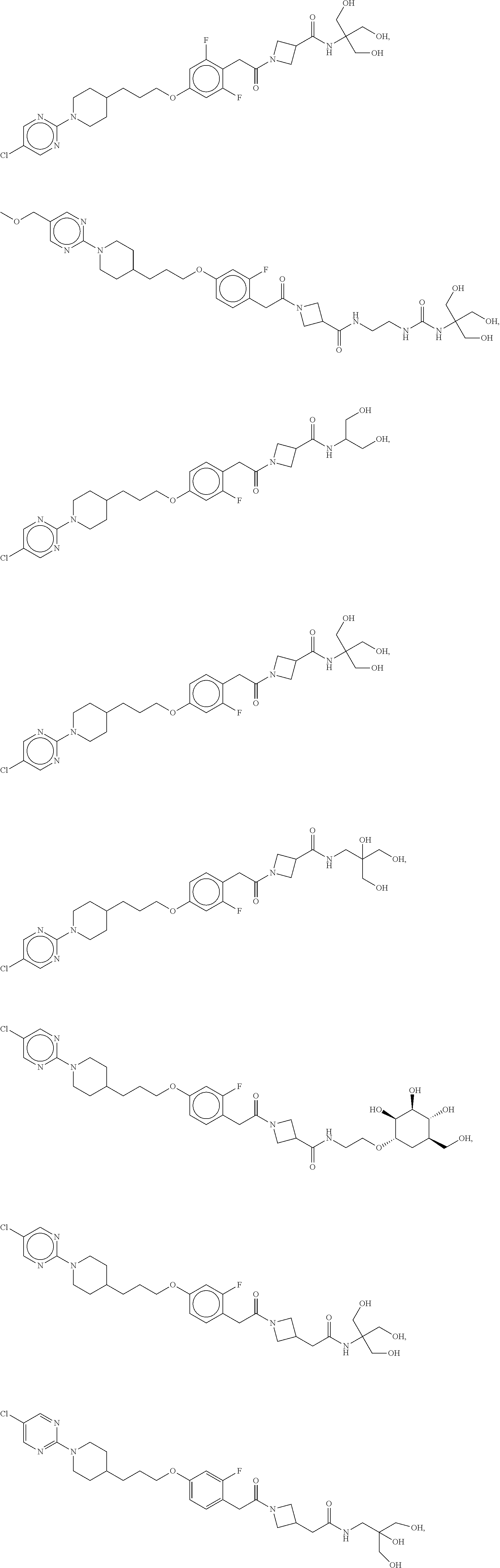 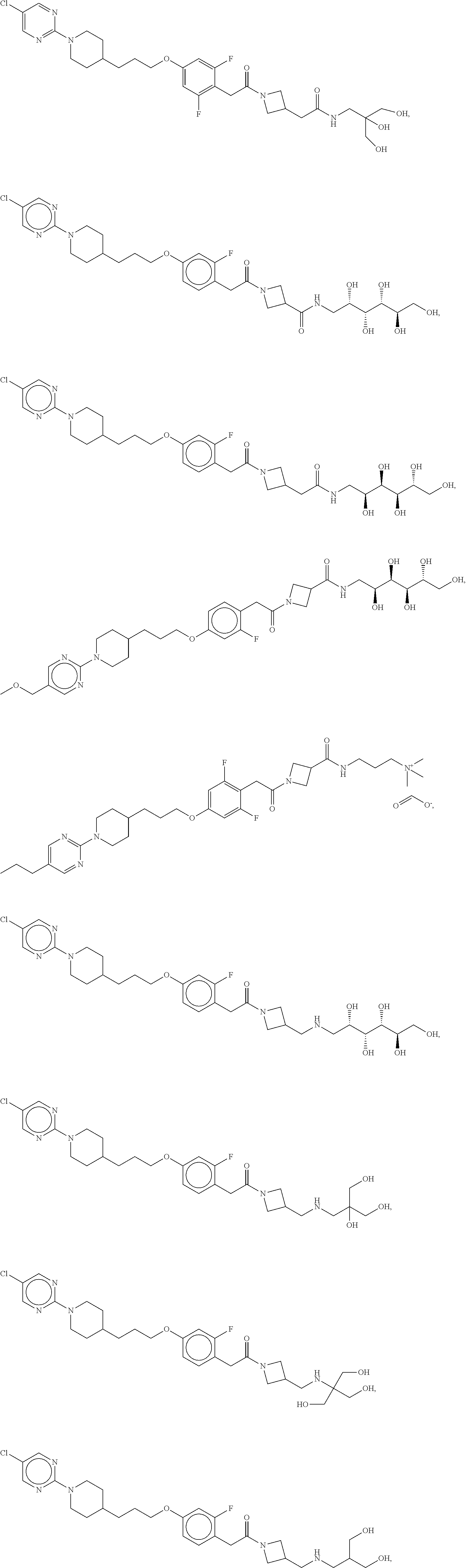 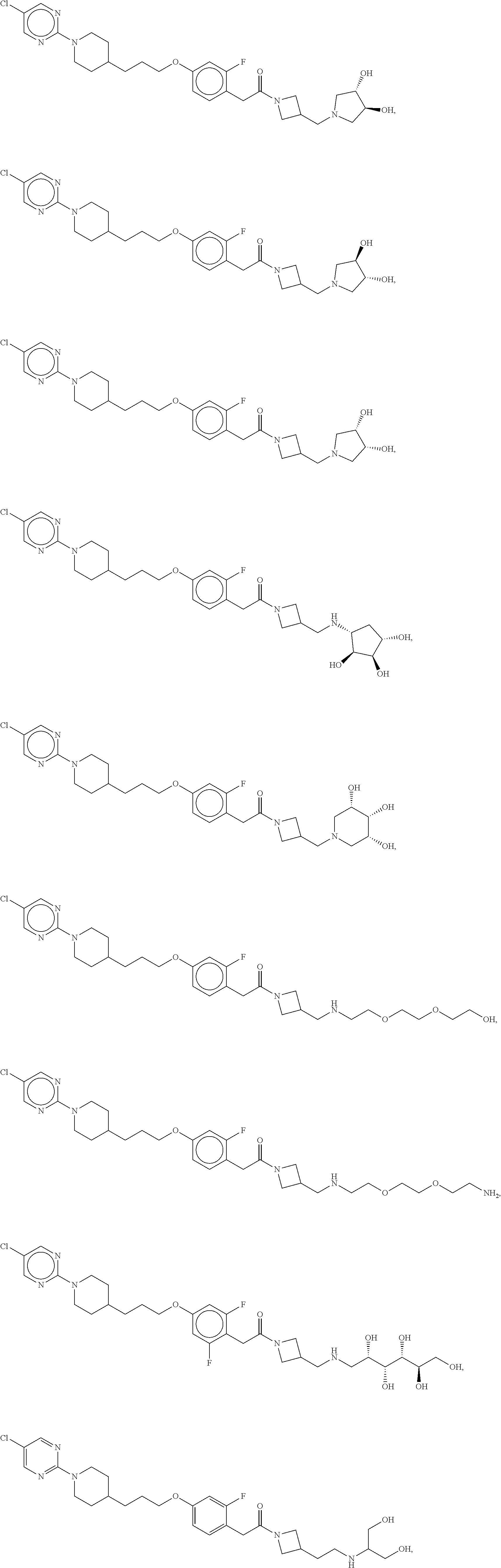 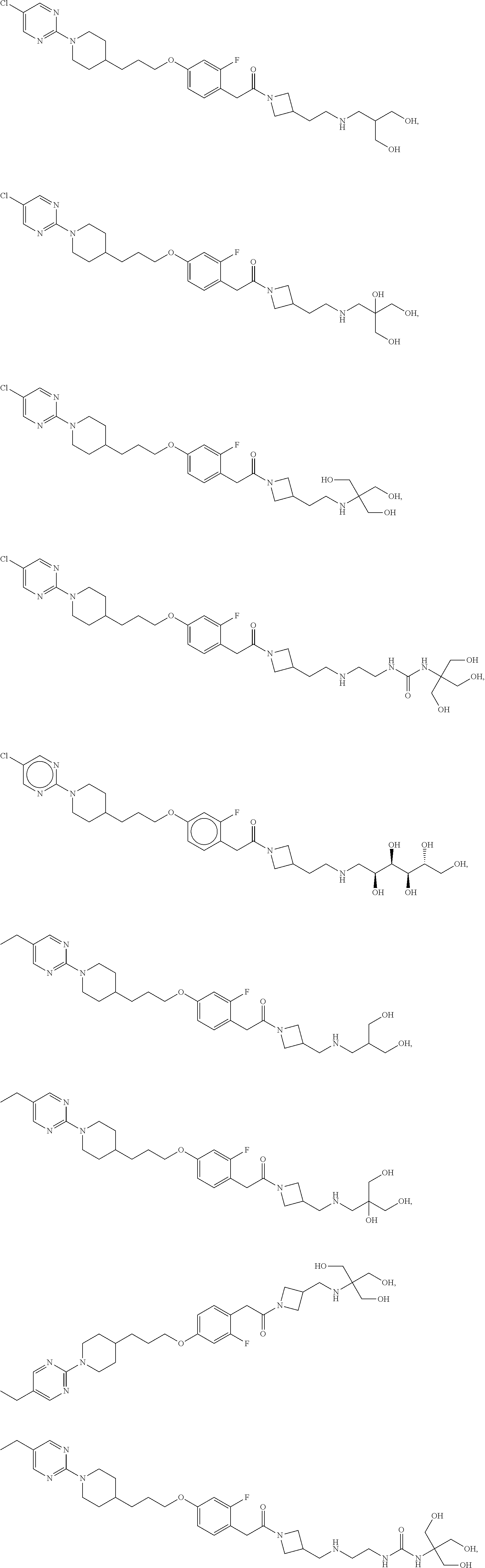 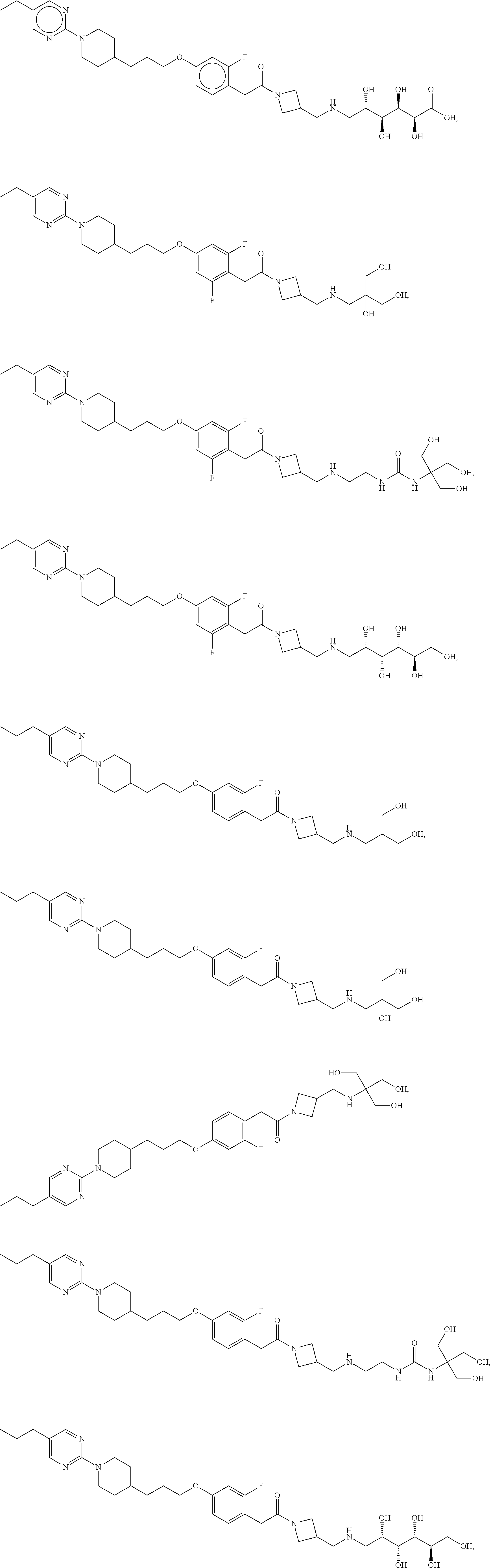 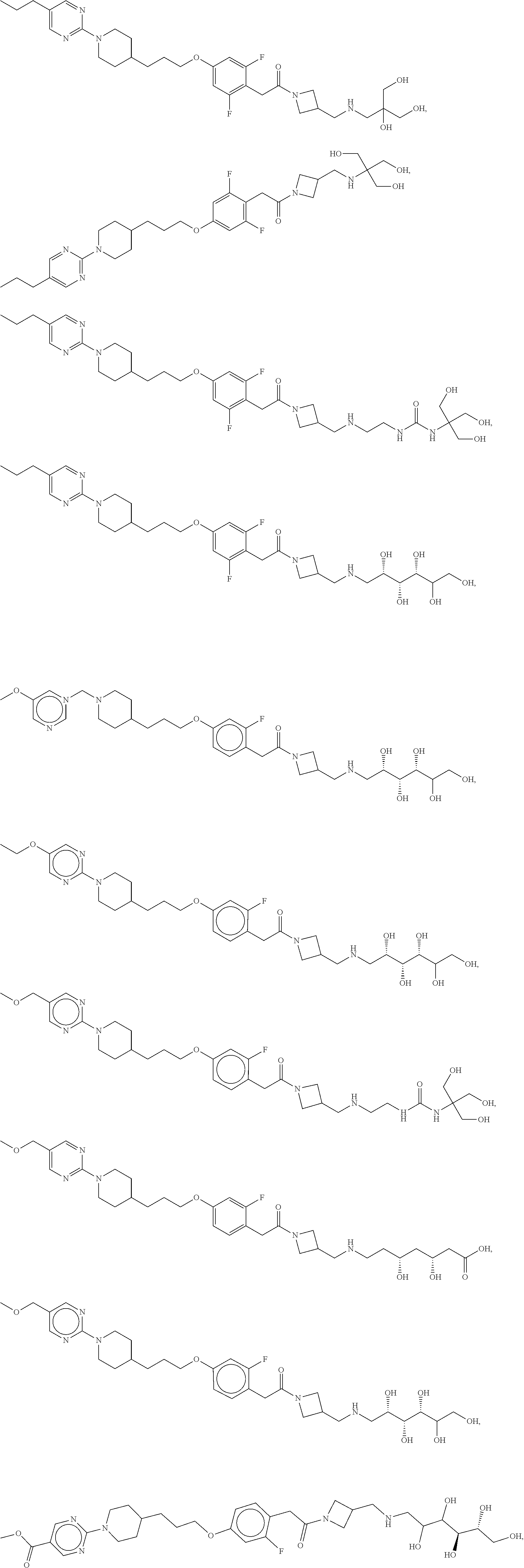 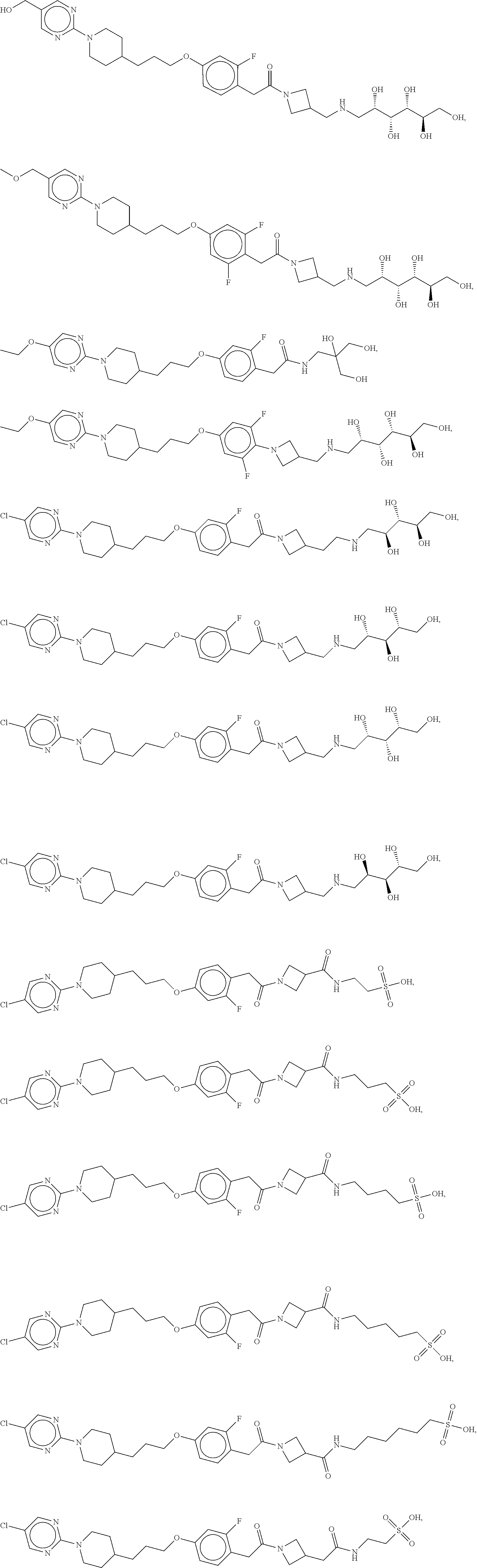 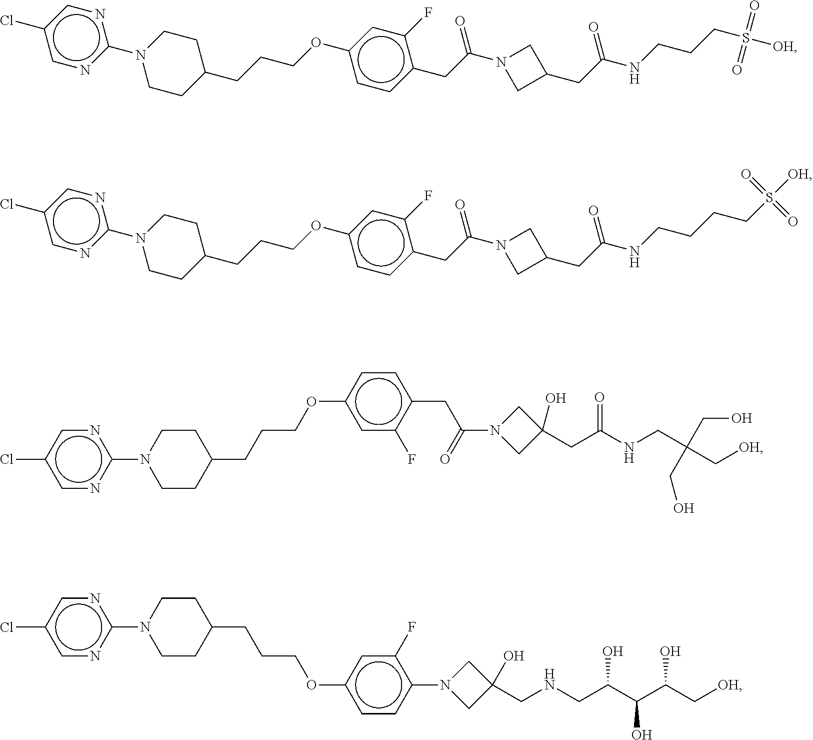
or a pharmaceutically acceptable salt, solvate, or stereoisomer thereof.

15. The method of any one of claims 1-14, wherein the compound of Formula (B) is: 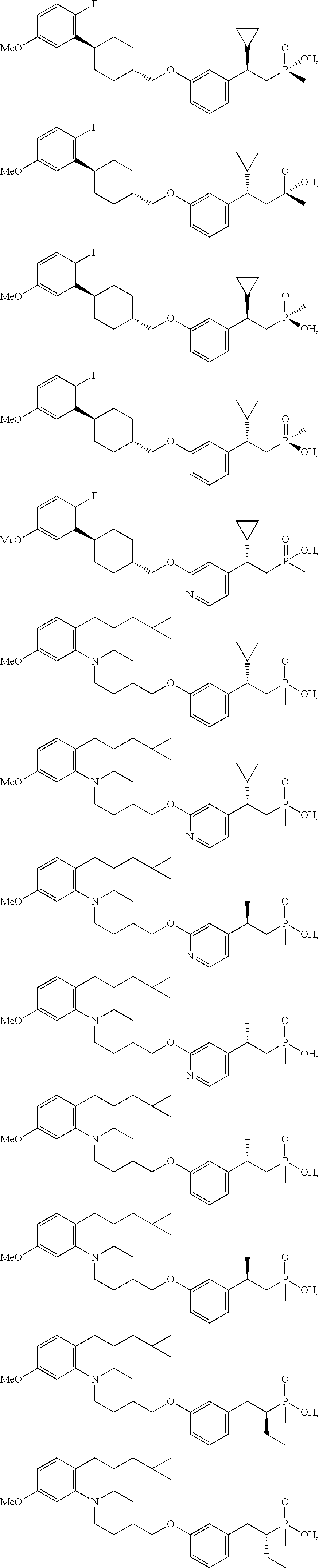
or a pharmaceutically acceptable salt, solvate, or stereoisomer thereof.

16. The method of any one of claims 1-15, further comprising administering one or more additional therapeutic agents to the individual in need thereof; wherein the one or more additional therapeutic agents are selected from: a TGR5 agonist, an SSTR5 antagonist, an SSTR5 inverse agonist, a CCK1 agonist, a PDE4 inhibitor, a DPP-4 inhibitor, a GLP-1 receptor agonist, a GOAT inhibitor, metformin, and combinations thereof.

17. The method of any one of claims 1-16, wherein the individual in need thereof is a human.

18. A pharmaceutical composition comprising:
i) a compound of Formula (A): 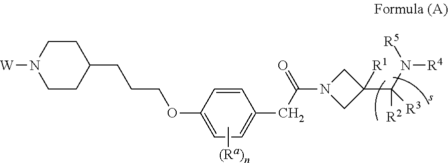
or a pharmaceutically acceptable salt, solvate, or stereoisomer thereof, wherein:
R¹ is hydrogen, —OH, or C₁₋₈ alkyl, wherein the alkyl is unsubstituted or substituted by —OH or —O(C₁₋₆ alkyl);
each R² and R³ is hydrogen;
or R² and R³ on the same carbon atom are taken together to form ═0;
R⁴ is hydrogen or C₁₋₆ alkyl;
R⁵ is C₁₋₈ alkyl, C₃₋₈ cycloalkyl, 4- to 8-membered heterocycloalkyl, —[(CH₂)ᵣ—Z]ₜ—R⁶, —[(CHR^{d})ᵣ—Z]ₜ—R⁶, or —[(C(R^{d})₂)ᵣ—Z]ₜ—R⁶; wherein each alkyl, cycloalkyl, and 4- to 8-membered heterocycloalkyl is substituted by 1-6 R^{c} groups
each Z is independently —CH₂O—, —CH₂NR^{d}, —CH₂N⁺(R^{d})₂—, or —NH—C(═O)—NH—;
each r is independently 1-6;
each t is independently 1-6;
R⁶ is hydrogen, C₁₋₈ alkyl, C₃₋₈ cycloalkyl, or 4- to 8-membered heterocycloalkyl, wherein the alkyl, cycloalkyl, or 4- to 8-membered heterocycloalkyl is unsubstituted or substituted by 1-6 R^{c} groups;
or R⁴ and R⁵ are taken together with the nitrogen to which they are attached to form a 4- to 8-membered heterocycloalkyl, which is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH) 2, —P(═O)(OH)(R^{d}), —P(═O)(OH)(OR^{d}), 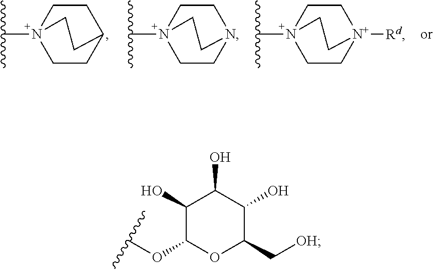
each R^{d} is independently C₁₋₆ alkyl;
each R^{a} is independently halogen, —CN, C₁₋₆ alkyl, C₁₋₆ fluoroalkyl, or C₃-6 cycloalkyl;
W is phenyl or 5-6 membered monocyclic heteroaryl, wherein the phenyl or heteroaryl is unsubstituted or substituted with 1, 2, or 3 substituents selected from R^{b};
each R^{b} is independently halogen, —OH, —CN, —C(O)OH, —C(O)O(C₁₋₆ alkyl), C₁₋₆ alkyl, C₁₋₆ alkoxy, C₃-6 cycloalkyl, phenyl, or 5- to 6-membered heteroaryl; wherein each alkyl, alkoxy, and cycloalkyl is unsubstituted or substituted with 1, 2, or 3 substituents selected from halogen, —OH, C₁₋₆ alkyl, and C₁₋₆ alkoxy;
n is 0-4; and
s is 1 or 2;
ii) a compound of Formula (B): 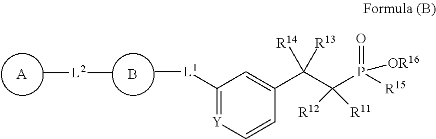
or a pharmaceutically acceptable salt, solvate, or stereoisomer thereof, wherein:
R¹¹, R¹², and R¹³ are each independently hydrogen, halogen, or C₁-C₆ alkyl;
R¹⁴ is hydrogen, C₁-C₆ alkyl, or C₃-C₆ cycloalkyl;
R¹⁵ is C₁-C₆ alkyl;
R¹⁶ is hydrogen or C₁-C₆ alkyl;
Y is CH or N;
L¹ is *—O—CH₂—, *—CH₂—O—, *—NR¹⁷—CH₂—, *—NR¹⁷—C(O)—, *—C(O)—NR¹⁷—, or *—C(O)—CH₂—;
wherein * represents the connection to Ring B;
R¹⁷ is hydrogen or C₁-C₆ alkyl;
Ring B is 3- to 6-membered heterocycloalkylene; wherein the heterocycloalkylene is unsubstituted or substituted with 1, 2, 3, or 4 R^{B} substituents;
or Ring B is C₃-C₆ cycloalkylene; wherein the cycloalkylene is unsubstituted or substituted with 1, 2, 3, or 4 R^{B} substituents;
each R^{B} is independently halogen, C₁-C₆ alkyl, or C₁-C₆ fluoroalkyl;
L² is a bond or C₁-C₆ alkylene; wherein the alkylene is unsubstituted or substituted with 1, 2, or 3 substituents selected from the group consisting of —OH, C₁-C₆ alkyl, and —O—(C₁-C₆ alkyl);
Ring A is aryl or heteroaryl; wherein the aryl or heteroaryl is unsubstituted or substituted with 1, 2, or 3 R^{A} substituents;
each R^{A} is independently —F, —Cl, C₁-C₁₀ alkyl, C₁-C₁₀ fluoroalkyl, C₁-C₁₀ hydroxyalkyl, —OH, —OR¹⁸, —(C₁-C₆ alkylene)—OR¹⁸, —N(R¹⁹)₂, —(C₁-C₆ alkylene)-N(R¹⁹)₂;
each R¹⁸ is independently C₁-C₆ alkyl; wherein each alkyl is independently unsubstituted or substituted with 1, 2, 3, 4, or 5 substituents selected from the group consisting of halogen, —OH, C₁-C₆ alkyl and C₁-C₆ hydroxyalkyl; and
each R¹⁹ is independently hydrogen, C₁-C₆ alkyl, or monocyclic heteroaryl; wherein each alkyl and heteroaryl is independently unsubstituted or substituted with 1, 2, 3, 4, or 5 substituents selected from the group consisting of halogen, —OH, C₁-C₆ alkyl and C₁-C₆ hydroxyalkyl;
or two R¹⁹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl; and
iii) a pharmaceutically acceptable excipient.
